(19) United States
(12) Patent Application Publication
(10) Pub. No.: US 2026/0248440 A1
(43) Pub. Date: Aug. 27, 2026

(54) GASTRIC MYOELECTRICAL SIGNATURE OF FEEDING READINESS IN PRETERM INFANTS

(71) Applicant: THE BOARD OF REGENTS OF THE UNIVERSITY OF TEXAS SYSTEM, Austin, TX (US)

(72) Inventors: Eric Brum Ortigoza, Dallas, TX (US); Hanli Liu, Arlington, TX (US)

(73) Assignee: THE BOARD OF REGENTS OF THE UNIVERSITY OF TEXAS SYSTEM, Austin, TX (US)

(21) Appl. No.: 19/547,284

(22) Filed: Feb. 23, 2026

Related U.S. Application Data
(60) Provisional application No. 63/761,376, filed on Feb. 21, 2025.

(51) Int. Cl. A61B 5/392; A61B 5/00; A61B 5/397

(52) U.S. Cl. CPC A61B 5/392; A61B 5/397; A61B 5/4836; A61B 5/725; A61B 2503/045

(57) ABSTRACT

Provided herein are methods of predicting and/or diagnosing mammalian infants with feeding intolerance or feeding tolerance based on electrogastrography data. Further provided herein are methods of distinguishing between developmental and pathological feeding intolerance in preterm infants. The methods disclosed herein may be used to inform on suitable treatment for preterm infants with feeding intolerance.

### CROSS REFERENCE TO RELATED APPLICATION

This application claims priority to U.S. Provisional Patent Application Ser. No. 63/761,376, filed Feb. 21, 2025, and titled “GASTRIC MYOELECTRICAL SIGNATURE OF FEEDING READINESS IN PRETERM INFANTS,” which is incorporated by reference herein in its entirety.

### ACKNOWLEDGEMENT OF GOVERNMENT SUPPORT

This invention was made with government support under Grant No. HD108443, awarded by the National Institutes of Health. The government has certain rights in the invention.

### BACKGROUND

### 1. Field

The present disclosure relates to the field of prediction, diagnoses, and treatment of feeding intolerance in a subject using electrogastrography and/or biomarkers.

### 2. Discussion of Related Art

Feeding intolerance is a prevalent issue among preterm infants, with studies indicating that approximately 40-50% of very low birth weight infants experience this condition. Due to the immaturity of their gastrointestinal (GI) systems, preterm infants are prone to developing feeding intolerance, which can lead to significant complications such as malnutrition and poor neurodevelopmental outcomes. Feeding intolerance is characterized by symptoms such as gastric residuals, abdominal distension, and vomiting. Prolonged feeding intolerance is associated with an increased risk of necrotizing enterocolitis (NEC), a life-threatening condition with a mortality rate of up to 30%. Additionally, feeding intolerance often results in disrupted nutrition, leading to postnatal growth failure, which affects up to 60% of preterm infants by hospital discharge.

Current clinical methods for assessing feeding intolerance rely primarily on direct observations and non-specific measures, such as abdominal circumference, gastric residual volume, and the presence of vomiting. However, these approaches are subjective and not reliable indicators of gastrointestinal maturity or feeding readiness. For example, the measurement of gastric residual volume can vary significantly, and there is no established threshold to determine when intervention is needed. Consequently, feeding practices often differ from one case to another, which can result in both underfeeding and overfeeding, further complicating the infant's health. Therefore, there is a pressing need to develop a non-invasive tool or method that can accurately assess gastrointestinal maturity and feeding readiness, allowing for better management of enteral feeding, reducing risks, and improving long-term developmental outcomes.

### SUMMARY

In some aspects, the current disclosure encompasses a method of predicting and/or diagnosing feeding intolerance (FI) in a subject in need thereof, the method comprising: (a) providing pre- and during feeding electrogastrography (EGG) signal data from at least one feed for the subject; (b) obtaining mean power ratios mPRdur/pre (mean power ratio of during and pre-feeding signal) from power spectral density (PSD) obtained from during and pre-feeding EGG signals; and (c) predicting/diagnosing feeding intolerance in the subject if the mPRdur/pre ratio is less than or equal to one. In some aspects, step (a) may further comprise providing post feeding EGG data and step (b) may further comprise obtaining a mPRpost/pre (mean power ratio of post/pre-feeding signal data). In some aspects, the method may further comprise comparing the mPRdur/pre ratio to reference values using bootstrap resampling with replacement to generate empirical confidence intervals. In some aspects, the bootstrap resampling comprises at least 1,000 iterations. In some aspects, the empirical confidence intervals are defined by the 2.5th and 97.5th percentiles of the bootstrap distribution. In some aspects, the EGG signal data is obtained from the subject within first 1-10 weeks after birth of the subject. In some aspects, EGG signal data is obtained from the subject within first 2 weeks after birth. In some aspects of the disclosed method, step (b) comprises obtaining power spectral density (PSD) value within one of more of specific frequency bands comprising Bradygastria (0.5-2 cpm), Normogastria (2-4 cpm), or Tachygastria (4-9 cpm). In some aspects, step (b) comprises obtaining power spectral density (PSD) over all of the specific frequency bands (0.5-9 cpm). In some aspects, the method further comprises preprocessing of the EGG data before performing step (b). Non-limiting examples of preprocessing of EGG data include, but are not restricted to down sampling the data to 500 Hz to obtain a down sampled time series; applying a third-order polynomial fit to detrend the data and capture temporal trends in the down sampled time series; subtracting the fitted trend from the down sampled time series to obtain a detrended time series; and filtering the detrended time series using a low-pass filter at any one or more of 0.3 Hz to 1 Hz to mitigate potential filtering-induced phase shifts. In some aspects, the detrended signal is low-pass filtered at 0.37 Hz (~22.2 cpm) using a zero-phase second-order Butterworth filter.

In some aspects, the PSD has a frequency resolution of less than 4 mHz. In some aspects, the current disclosure also encompasses classifying subjects into types of FI, for example NFI (no feeding intolerance), pathological FI (PFI) or developmental FI (DFI). In some aspects, the subject is a mammal. In some aspects, the subject is a preterm infant.

In some aspects, the current disclosure also encompasses a method of treating a preterm infant in need thereof for a feeding intolerance (FI) comprising: determining a mean power ratio mPRdur/pre using the steps of (a) providing pre- and during feeding electrogastrography (EGG) signal data from at least one feed for the preterm infant, and (b) obtaining mean power ratios mPRdur/pre from power spectral density (PSD) obtained from during and pre-feeding EGG signals; and administering a treatment to the preterm infant, wherein the preterm infant has been determined to have the mPRdur/pre ratio of less than or equal to one. In some aspects, step (a) further comprises providing post feeding EGG data and obtaining a mPRpost/pre (mean power ratio of post/pre-feeding signal data). In some aspects, the treatment may vary based on the diagnoses and the severity of the condition. Non-limiting examples of options for treating FI include, but are not limited to minimal enteral nutrition (MEN), slow advancement of feeds, use of human milk, fortified feeds, parenteral nutrition, hydrolyzed or elemental formulas, prokinetic agents, upright or lateral positioning, probiotics, avoidance of unnecessary antibiotics, gastric residual monitoring, continuous vs. bolus feeding adjustments, abdominal massage, non-nutritive sucking, thickened feeds for reflux, acid suppressants in severe gastroesophageal reflux disease (GERD), bowel rest (NPO), intravenous fluids, broad-spectrum antibiotics, and surgical intervention for necrosis or obstruction, or any combination thereof. In some aspects, EGG signal data is obtained within first 1-10 weeks after birth of the preterm infant. In some aspects, step (b) comprises obtaining power spectral density (PSD) value within one of more of specific frequency bands comprising Bradygastria (0.5-2 cpm), Normogastria (2-4 cpm), and Tachygastria (4-9 cpm). In some aspects, step (b) comprises obtaining power spectral density (PSD) over all of the specific frequency bands (0.5-9 cpm). In some aspects, the method further comprises preprocessing of the EGG data before step (b). Non-limiting example of preprocessing steps include, but are not limited to down sampling the data to 500 Hz to obtain a down sampled time series; applying a third-order polynomial fit to detrend the data and capture temporal trends in the down sampled time series; subtracting the fitted trend from the down sampled time series to obtain a detrended time series; filtering the detrended time series using a low-pass filter at any one or more of 0.5 Hz to 1 Hz to mitigate potential filtering-induced phase shifts; and any combination thereof. In some aspects, the PSD has a frequency resolution of less than 4 mHz.

In some aspects, the current disclosure also encompasses a computer implemented method for predicting/diagnosing feeding intolerance (FI) in a subject in need thereof, the method comprising: (a) providing pre- and during feeding electrogastrography (EGG) signal data from at least one feed for the subject; (b) obtaining mean power ratios mPRdur/pre from power spectral density (PSD) obtained from during and pre-feeding EGG signals; (c) predicting/diagnosing feeding intolerance in the subject if the mPRdur/pre ratio is less than or equal to one, or (d) predicting/diagnosis no feeding intolerance (NFI) or feeding tolerance (FT) in the subject if the mPRdur/pre ratio is greater than one. In some aspects, step (a) further comprises providing post feeding EGG data and obtaining a mPRpost/pre (mean power ratio of post/pre-feeding signal data).

In some aspects, the current disclosure also encompasses a system for predicting/diagnosing feeding intolerance (FI) in a subject, the system comprising: (a) an EGG machine; (b) a memory; and (c) a processor operable to execute instructions stored in the memory to determine mean power ratios mPRdur/pre from power spectral density (PSD) obtained from during and pre-feeding EGG signals from the subject.

### BRIEF DESCRIPTION OF THE DRAWINGS

The following drawings form part of the present specification and are included to further demonstrate certain aspects of the present disclosure. Aspects of the present disclosure may be better understood by reference to one or more of these drawings in combination with the detailed description of specific aspects presented herein.

FIGS. 1A-1B provide the electrogastrography (EGG) electrode configuration. FIG. 1A shows the typical configuration of an enteral feeding tube commonly employed in neonatal intensive care settings. FIG. 1B shows the arrangement of three EGG electrodes: the negative electrode (indicated by ‘1 (−)’) is positioned in the upper left quadrant, close to the mid-clavicular line. The positive electrode (marked as ‘2 (+)’) is situated midway between the base of the breastbone and the navel, slightly below the negative electrode's level. The ground electrode (identified as ‘3 (g)’) is positioned at the mid-axillary line, beneath the left rib margin.

FIG. 2 provides a flow chart showing the process of averaging data segments to form cohesive representations for pre-feeding, during-feeding, and post-feeding phases. Averaging Pre 1 and Pre 2 results in a single representation for the pre-feeding phase, and a similar approach is applied to derive representations for during-feeding and post-feeding phases.

FIG. 3 provides a flow chart showing the sequential steps from acquired raw EGG (Electrogastrogram) data to various calculations and analyses performed in the study. Each step represents a stage in the data processing pipeline, demonstrating the systematic approach employed in the analysis.

FIG. 4 provides the averaged power spectral density (PSD) for different feeding phases. Curves (a), (b), and (c) represent the averaged PSD over 75 NFI (No Feeding Intolerance) babies for pre-feeding, during-feeding, and post-feeding periods, respectively. Similarly, curves (d), (e), and (f) illustrate the corresponding calculations for 9 FI (Feeding Intolerance) babies.

FIGS. 5A-5B provide the averaged ratio curves derived from the calculated dur/pre and post/pre ratios for each baby. FIG. 5A, provides the averaged Rdur/pre ratio curves for NFI (No Feeding Intolerance) and FI (Feeding Intolerance) groups are presented. FIG. 5B illustrates the averaged Rpost/pre ratio curves for NFI and FI groups. The vertical lines represent the standard error of the mean.

FIG. 6A is a graph showing the averaged mPSD values at each frequency band between NFI (blue bars) and FI (red bars) groups during the pre-feeding phase. Followed by the corresponding p-values obtained from the t-tests at each frequency band (P-Value at Brady Band=0.61, P-Value at Normal Band=0.61, P-Value at Tachy Band=0.73).

FIG. 6B is a graph showing the averaged dur/pre ratio values within each group at each frequency band, with accompanying p-values from the t-tests for NFI vs FI comparisons (P-Value at Brady Band=0.027, P-Value at Normal Band=0.069, P-Value at Tachy Band=0.038).

FIG. 6C is a graph showing the same analysis as FIG. 6B, but for post/pre ratio values.

FIG. 7A is a schematic showing the different forms of FI studied.

FIG. 7B is a schematic providing the timeline for the study encounters.

FIGS. 8A-8B provide the averaged ratio curves derived from the calculated dur/pre and post/pre ratios for each baby. FIG. 8A shows that the mean power ratio during/pre-feeding (mPRdur/pre) is consistently >1 for NFI compared to DFI and PFI, which are mostly ≤1, suggesting normal motility for NFI and dysmotility for DFI and PFI. FIG. 8B, shows that the mPRdur/pre measure can differentiate infants with NFI vs. FI (combined DFI+PFI) during first 2 weeks of life. At frequency ranges 0.7-3.5 cpm, FI babies have a mPRdur/pre ≤1 and NFI babies >1, suggesting more dysmotility of the FI babies. Ratios above orange line=good motility, at or below orange line=dysmotility. NFI=No Feeding Intolerance, PFI=Pathologic Feeding Intolerance, DFI=Developmental Feeding Intolerance, FI=Feeding Intolerance (combined DFI+PFI), cpm=cycles per minute, SEM=Standard Error of the Mean.

FIGS. 9A-9C provide the median and variance differential analysis shown as volcano plots: FIG. 9A: shows that NFI group demonstrated lower (a-D-mannosyl)2-b-D-mannosyl-N-acetylglucosamine (p<0.001), acetylaminoadipate (p<0.001), and lacto-N-triaose (p<0.001) when compared to the DFI group. FIG. 9B: shows that NFI group demonstrated higher alpha-tocopherol (p<0.001), gamma-tocopherol (p<0.001), and 24-hydroxycholesterol (p<0.001) when compared to the PFI group. FIG. 9C: shows that DFI group demonstrated higher alpha-tocopherol (p=0.0105) and lower galactosylglycerol (p=0.001) compared to the PFI group.

FIGS. 10A-10C show the distributions of bootstrapped mean differences in mPSD values for the pre-feeding period between the NFI (n=75) and FI (n=9) groups. Each panel (a-c) corresponds to one frequency band: FIG. 10A: bradygastria, FIG. 10B: normogastria, and FIG. 10C: tachygastria. The x-axis shows the mean difference values, and the y-axis shows the bootstrap sample counts (visualized as histograms). The red dashed lines mark the 2.5th and 97.5th percentiles of the bootstrap distribution, while the blue dashed line indicates zero mean difference.

FIGS. 11A-11F provides bootstrapped distributions of mean differences in mPR values between NFI (n=75) and FI (n=9) groups for each gastric frequency band. FIG. 11A shows the bootstrapped distribution of mean differences for mPRDur/Pre in the bradygastria band (0.5-2 cpm). The x-axis represents the mean difference values, and the y-axis represents the bootstrap sample counts. The red dashed lines indicate the 2.5th and 97.5th percentiles of the bootstrap distribution, and the blue dashed line indicates zero mean difference. FIG. 11B shows the bootstrapped distribution of mean differences for mPRDur/Pre in the normogastria band (2-4 cpm), with axes and percentile markings as described for FIG. 11A. FIG. 11C shows the bootstrapped distribution of mean differences for mPRDur/Pre in the tachygastria band (4-9 cpm), with the same axis conventions and percentile indicators. FIG. 11D shows the bootstrapped distribution of mean differences for mPRPost/Pre in the bradygastria band. The x- and y-axes correspond to mean differences and bootstrap counts, respectively, with red dashed percentile boundaries and a blue zero-difference reference line. FIG. 11E shows the bootstrapped distribution of mean differences for mPRPost/Pre in the normogastria band, following the same visual conventions noted for FIG. 11D. FIG. 11F shows the bootstrapped distribution of mean differences for mPRPost/Pre in the tachygastria band, again using the same axis labels and statistical threshold indicators.

The drawing figures do not limit the present disclosure to the specific aspects disclosed and described herein. The drawings are not necessarily to scale, emphasis instead being placed on clearly illustrating principles of certain aspects of the present disclosure.

### DETAILED DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized and changes can be made without departing from the scope of the present disclosure. The following description is, therefore, not to be taken in a limiting sense.

Enteral feeding is challenging in preterm infants because of gastrointestinal (GI) immaturity. Feeding intolerance affects 75% of very low birth weight preterm infants, leading to malnutrition and poor neurodevelopmental outcomes. Early signs and symptoms of Feeding Intolerance, for instance Developmental Feeding Intolerance (DFI) and Pathologic Feeding Intolerance (PFI) are hard to diagnose. Electrogastrography (EGG) is a non-invasive technology that measures gastric myoelectrical activity and can be utilized to measure changes that occur with maturation at different gestational ages (GA). Three gastric rhythms (GR) exist between 0.5-9 cycles per minute (cpm), namely, bradygastria (0.5≤GR<2 cpm), normogastria (2≤GR<4 cpm), and tachygastria (4≤GR<9 cpm). In some aspects, the current disclosure is based on efforts to use EGG-derived parameters to diagnose/predict feeding intolerance (FI) and/or non-feeding intolerance (NFI), also referred to as feeing tolerance (FT) in preterm babies. Pre-, during, and post-feed data were analyzed. Unexpectedly, it was found that the mean power ratios (mPR) between during- and pre-feeding periods (mPRdur/pre) can be indicative of gastric motility and status of enteral feeding.

In some aspects, the current disclosure also provides additional non-invasive diagnostic and treatment methods using comparative fecal metabolomics, for use either in isolation or in combination with EGG to further predict/diagnose and/or treat feeding intolerance in preterm infants. Data presented in the instant disclosure provide surprising results showing that fecal Vitamin E is lower in infants with PFI.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

The terms “comprising,” “including,” and “having” are used interchangeably in this disclosure. The terms “comprising,” “including,” and “having” mean to include, but not necessarily be limited to the things so described.

The terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); Chambers Dictionary of Science and Technology (Walker ed., 1999); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

As used herein, “individual,” “subject,” “host,” and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject is a preterm infant. In some aspects, the subject is a human. In other aspects, the subject is a human infant in need of diagnosing/predicting and/or treating a feeding intolerance.

In some aspects, the current disclosure encompasses a method of predicting and/or diagnosing feeding intolerance (FI) in a subject in need thereof, the method comprising: (a) providing pre- and during feeding electrogastrography (EGG) signal data from at least one feed for the subject; (b) obtaining mean power ratios mPRdur/pre (mean power ratio) from power spectral density (PSD) obtained from during and pre-feeding EGG signals; and (c) predicting/diagnosing feeding intolerance in the subject if the mPRdur/pre ratio is less than or equal to one.

In an aspect, the term “standard value” or “reference range” or “reference levels” refer to predefined values or ranges of values used as benchmarks to interpret the results of an assay. These levels are crucial for distinguishing between normal and abnormal results, thereby aiding in the diagnosis of a specific condition or disease. In an aspect, the terms refer to specific values or ranges that are considered normal or typical for a given population. These levels serve as a baseline for comparison or a standard against which individual test results can be compared, facilitating the identification of deviations that may indicate a disease or condition. These values and ranges are typically obtained from studies on a healthy population to determine the normal range of values. Statistical methods are typically used to define the reference range, often encompassing the central 95% of the values obtained from the healthy population (mean±2 standard deviations). Age, gender, ethnicity, and other demographic factors can affect reference levels. Natural biological variability among individuals can also lead to differences in reference levels. Differences in assay methods, sensitivities, reagents, and equipment can also influence the reference levels. Reference levels are used to interpret assay results, helping clinicians determine whether a patient's result is within the normal range or indicative of a potential health issue. Specific cut-off values may be established within the reference levels to guide clinical decisions, such as initiating further diagnostic testing or treatment. As will be appreciated in the art, once the “standard level” or “reference range” is known, it can be used repeatedly as a standard for comparison. Though typically standard value may be determined based on a population, in an aspect, a standard value may also refer to one or more values obtained from samples obtained from the subject, at a time prior to the sample being assayed.

### II. Electrogastrographical Methods to Determine Feed Intolerance (FI)

### A. Feeding Intolerance

Feeding intolerance (FI) is a common challenge in neonates, particularly preterm infants, and is characterized by the inability to tolerate enteral feeding due to gastrointestinal immaturity or underlying pathology. Studies have reported that approximately 27.5% of preterm infants experience FI, with the incidence rising to 76.4% among VLBW infants. The most common manifestation observed is simple gastric retention, accounting for 47.2% of cases, typically presenting within the first three days after initiating feeding. It manifests through symptoms such as gastric residuals, abdominal distension, vomiting, and delayed gastric emptying, often leading to disruptions in nutritional intake and growth. The causes of FI can be developmental, arising from immature gut motility and enzyme function, or pathological, resulting from conditions like necrotizing enterocolitis (NEC), infections, or congenital anomalies. Management involves careful monitoring, gradual feeding advancements, and, in some cases, specialized formulas or parenteral nutrition to ensure adequate caloric intake while minimizing gastrointestinal distress. Early identification and appropriate intervention are essential to prevent complications and support optimal growth and development in affected infants.

There are several types of feeding intolerance observed in preterm babies. One common type is delayed gastric emptying, where the stomach takes longer than usual to empty its contents. Another type is immature gut motility, which refers to the lack of coordinated muscle contractions in the intestines that move food through the digestive tract. Poor coordination of feeding reflexes, such as sucking and swallowing, is also a significant factor. Additionally, complications like infections or oxygen deficiency can exacerbate feeding intolerance. Broadly speaking, FI can be of developmental and pathological types, each with distinct underlying causes. Developmental FI is primarily due to the immaturity of the gastrointestinal (GI) system, commonly seen in preterm infants whose digestive and absorptive functions are underdeveloped. The immaturity of gastric motility, delayed gastric emptying, and inefficient coordination between sucking, swallowing, and breathing contribute to difficulties in feeding tolerance. Additionally, the underdeveloped gut barrier and enzyme systems may lead to difficulties in digesting and absorbing nutrients, exacerbating FI. In most cases, developmental FI improves as the infant matures and the digestive system gains functional competence, though careful feeding advancements and supportive care are essential to minimize complications.

Pathological FI, in contrast, is associated with underlying medical conditions that disrupt normal feeding processes and gastrointestinal function. Common causes include necrotizing enterocolitis (NEC), infections, congenital abnormalities such as intestinal atresia or malrotation, and metabolic disorders that affect digestion and absorption. Unlike developmental FI, which resolves with time, pathological FI often requires medical intervention, such as parenteral nutrition, specialized formulas, or surgical correction of anatomical defects. Infections or inflammatory responses in the gut may further compromise feeding tolerance, necessitating close monitoring and targeted treatment. Differentiating between developmental and pathological FI is critical in neonatal care, as early recognition and appropriate management can significantly impact an infant's growth, nutritional status, and long-term health outcomes.

In some aspects, the current disclosure encompasses a EGG based methodology to distinguish between infants with no feeding intolerance (NFI), also referred to as feeding tolerance (FT), and infants with FI. In some aspects, the methods disclosed herein may be further used to distinguish between DFI and PFI. Information from the disclosed method can be used to predict, diagnose and inform on suitable treatment strategies for the subject.

Treatment of FI can vary based on the type and severity of the FI. A medical professional will be able to determine the best treatment course. In some aspects, the type of treatment used may be determined by the outcome of the disclosed method. Typically, treatment of FI may comprise one or more of minimal enteral nutrition (MEN), slow advancement of feeds, use of human milk, fortified feeds, parenteral nutrition, hydrolyzed or elemental formulas, prokinetic agents, upright or lateral positioning, probiotics, avoidance of unnecessary antibiotics, gastric residual monitoring, continuous vs. bolus feeding adjustments, abdominal massage, non-nutritive sucking, thickened feeds for reflux, acid suppressants in severe gastroesophageal reflux disease (GERD), bowel rest (NPO), intravenous fluids, broad-spectrum antibiotics, surgical intervention for necrosis or obstruction, or any combination thereof.

### B. Electrogastrography

In some aspects, the disclosed method uses EGG to predict, diagnose and/or distinguish between NFI and FI in infants. In some aspects, EGG signals utilized in the methods and systems of the present disclosure comprise cutaneous or skin-surface recordings obtained from electrodes placed on the abdominal skin. In some aspects, the EGG signals are not limited to cutaneous or skin-surface recordings and may additionally or alternatively be obtained using internal and/or invasive EGG systems, including signals acquired from electrodes positioned within the body, for example on or near the stomach, within the gastrointestinal tract, or otherwise internal to the subject (e.g., via implantable, endoluminal, catheter-based, endoscopic, laparoscopic, or surgical electrode configurations). Regardless of the manner of acquisition, the EGG signals may be processed using the same or similar signal processing and analysis techniques described herein

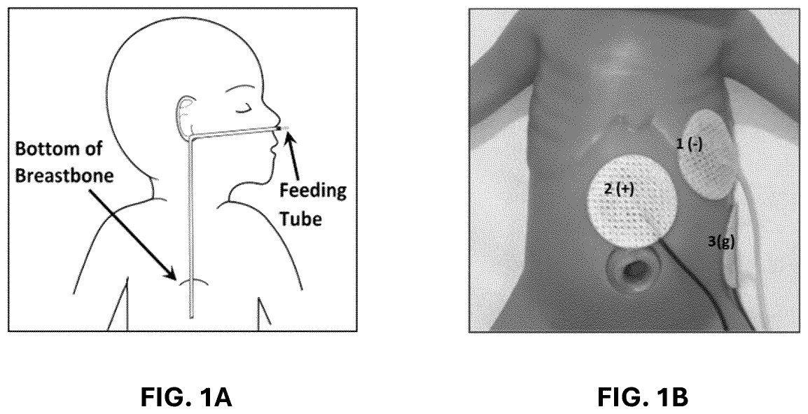

In some aspects, the system for performing electrogastrography diagnosis comprises an array of skin-surface electrodes arranged in a predefined configuration over the epigastric region to capture gastric myoelectrical signals. In some aspects, at least, or equal to, or at most, one, or two, or three, or four, or five, or more electrodes may be used. In some aspects, the disclosed method uses at least 3 electrodes. The electrodes may be placed anywhere over the infant's gut. An exemplary placement is provided in FIG. 1. The method for electrogastrography-based diagnosis begins with subject preparation. In some aspects, a conductive gel is applied to the electrode sites to reduce skin impedance, and electrodes are positioned in a standardized configuration over the gastric region. Baseline, or pre-prandial, EGG recording is then performed while the subject is in a resting position for at least about 15, 20, 25, 30, 35, 40, 45, 50, 55, or 60, or more minutes to establish a baseline gastric rhythm. This provides the pre-feeding signal. Following this, the subject may be fed a preparation, for example, milk to stimulate gastric activity, and the system records EGG signals for 15, 20, 25, 30, 35, 40, 45, 50, 55, or 60, or more minutes to collect signal during feeding. Optionally, in some aspects, post-feeding signal may also be collected for 15, 20, 25, 30, 35, 40, 45, 50, 55, or 60, or more minutes after completion of feeding. In some aspects, data may be collected for a single feed. In some aspects, data may be collected for multiple feed, for example at least one, at least 2, at least 3, at least 4, at least 5, at least 6, or more feeds for a subject in need thereof. In some aspects, the data may be collected over 1, 2, 3, 4, 5, 6, 7, 8, 9, 10 or more weeks after birth.

In some aspects, the duration of an EGG recording session and the number of feeding cycles captured are not limited to any particular length or count. For example, while certain exemplary recording sessions described herein may span approximately six hours and include two feeding cycles, in other aspects recordings may be shorter or longer (e.g., minutes to hours) and may include any number of feeding cycles (e.g., one feeding cycle, two feeding cycles, or more than two feeding cycles). In some aspects, the EGG signal data are recorded for a duration sufficient to capture at least a pre-feeding segment and a during-feeding segment for at least one feed, and optionally a post-feeding segment, and the disclosed analyses (including computation of PSD metrics and mean power ratios) may be performed on data from any one or more feeds (including analysis of a single feed, multiple feeds separately, and/or averaging, aggregating, or otherwise combining results across multiple feeds).

In some aspects, a high-gain amplifier coupled with an adaptive filtering unit enhances weak gastric slow-wave signals while minimizing interference from cardiac, respiratory, and motion artifacts. In some aspects, a microcontroller or computer-based data acquisition unit receives, digitizes, and stores EGG signals for further processing. A computational module executes algorithms for frequency analysis, noise reduction, spectral power estimation, and classification of abnormal gastric rhythms. In some aspects, a graphical user interface facilitates real-time monitoring, visualization, and report generation.

In some aspects, the recorded signals undergo processing and feature extraction. Typically, noise reduction techniques, including adaptive filtering and wavelet-based denoising, eliminate non-gastric artifacts. Spectral estimation using Fast Fourier Transform (FFT) or Short-Time Fourier Transform (STFT) extracts dominant gastric slow-wave frequencies. The amplitude and power analysis assesses signal intensity variations pre-, during, and post-meal. Time-domain analysis evaluates slow-wave propagation characteristics. Diagnostic classification and interpretation involve comparing during and pre-prandial EGG results to identify pathophysiological abnormalities. A machine learning-based classifier or rule-based expert system determines whether the extracted features indicate normal or abnormal gastric motility. A diagnostic report is generated with visualizations of EGG waveforms, frequency spectra, and statistical findings, which a clinician reviews to confirm the diagnosis and recommend treatment options.

In some exemplary aspects, the EGG data may be preprocessed using one or more of the steps of: down sampling the data to 500 Hz to obtain a down sampled time series; applying a third-order polynomial fit to detrend the data and capture temporal trends in the down sampled time series; subtracting the fitted trend from the down sampled time series to obtain a detrended time series; filtering the detrended time series using a low-pass filter at 0.3 Hz to 1 Hz to mitigate potential filtering-induced phase shifts. Examples of suitable software are disclosed herein and include, but are not limited to MATLAB, LabChart (ADInstruments), BioPac AcqKnowledge, EEGLAB (MATLAB Toolbox), Python (SciPy, NumPy, MNE-Python), custom-built software (C++, Python, or MATLAB-based), OriginPro, Spike2 (CED).

In some aspects, the low-pass filter may be applied at a frequency in the range of about 0.3 Hz to about 1 Hz. In some aspects, the low-pass filter frequency may be selected from about 0.3 Hz, about 0.35 Hz, about 0.37 Hz, about 0.4 Hz, about 0.45 Hz, about 0.5 Hz, about 0.55 Hz, about 0.6 Hz, about 0.65 Hz, about 0.7 Hz, about 0.75 Hz, about 0.8 Hz, about 0.85 Hz, about 0.9 Hz, about 0.95 Hz, or about 1 Hz. The selection of the low-pass filter frequency may depend on the specific application and the frequency content of interest in the gastric myoelectrical signals. Lower frequencies within this range may be preferred when it is desirable to attenuate higher-frequency noise while preserving the gastric slow-wave signals, which typically fall within the 0.5-9 cpm range.

In a specific aspect, the detrended signal is low-pass filtered at about 0.37 Hz (approximately 22.2 cpm) using a zero-phase second-order Butterworth filter. The use of a zero-phase filter configuration ensures that phase distortion is avoided, which is important for accurate temporal analysis of gastric myoelectrical activity. The Butterworth filter design provides a maximally flat frequency response in the passband, thereby minimizing signal distortion while effectively attenuating high-frequency noise components. In some aspects, Other low-pass designs may be employed-including Bessel (for maximally flat group delay), Chebyshev Type I/II, elliptic, or finite impulse response (FIR) filters (e.g., Kaiser-window or equiripple designs), provided that the filter characteristics are suitable for preserving the gastric slow-wave frequency content of interest.

In some aspects, the preprocessed data may be partitioned based on the feed timestamps prior to further processing. For example, the preprocessed data may be divided into pre-feeding, during and post-feeding signals and used to calculate power spectral density (PSD) and spectral means. A normal gastric rhythm is typically within 2.5 to 3.5 cycles per minute, while bradygastria, indicative of delayed gastric emptying, is less than 2.5 cycles per minute, and tachygastria, indicative of functional dyspepsia, exceeds 3.5 cycles per minute. In some aspects, the PSD value can be obtained within one of more of specific frequency bands comprising bradygastria (0.5-2 cpm), normogastria (2-4 cpm), or tachygastria (4-9 cpm). In some aspects, the PSD comprises obtaining values over all of the specific frequency bands (0.5-9 cpm).

In some exemplary aspects, the preprocessed signal may be further processed the following processing routines to obtain a mean power ratios mPRdur/pre from power spectral density (PSD):

Step 1: The power spectral density (PSD) across the entire gastric frequency band (0.5-9 cpm) during the feeding periods may be computed using a suitable software. In some aspects, this analysis may be conducted with the down-sampled frequency of 500 Hz, employing a 4-minute window and 2-minute overlap for each subject. The choice of the window and overlap can be varied by a person of ordinary skill in the art to obtain a desired resolution. For example, the utilization of a 4-minute window results in a frequency resolution of 0.004 Hz (or 4 mHz) in the PSD calculation, particularly between 0 to 1 Hz.

Step 2: In some aspects, feed data for each time stamp (pre-, during, and post feed) from more than one feed may be averages. For example, for data obtained over 2 feeds, quantification of mean PSD values over three sub-feeding periods may be obtained by averaging them between Pre-feed 1 and Pre-feed 2, During-feed 1 and During-feed 2, and Post-feed 1 and Post-feed 2.

Step 3: In some aspects, following the acquisition of the three-power spectral density (PSD) curves representing the pre-, during-, and post-feeding phases for a subject, the mean PSD curves for each phase may be calculated. After obtaining the power spectral density (PSD) values for the during-feeding and post-feeding periods two or more PSD ratio curves may be computed. These may be derived, for example, by dividing the PSD values during feeding and after feeding by pre-feeding phase, thus yielding two distinct PSD ratio curves, representing the relative changes in gastric activity during and after feeding compared to the pre-feeding baseline.

Step 4: In some aspects of the disclosed method, further analysis may be used to compare the ratios obtained from the subject data to a baseline ratio, or a ratio obtained from a non-patient, and/or a patient population to diagnose NFI, DFI or PFI. In an exemplary aspect an infant having been determined to have the mPRdur/pre ratio of less than or equal to one may be determined to have a FI. In some aspects, the disclosed steps may be performed as a single computer-based implementation or subdivided into routines for multiple implementations.

In some aspects, the disclosed method further comprises comparing the mPRdur/pre ratio to reference values using a nonparametric bootstrap resampling procedure with replacement to generate empirical confidence intervals. Bootstrap resampling is particularly advantageous when comparing groups with unequal sample sizes, as it allows estimation of empirical confidence intervals under data imbalance without requiring assumptions about underlying distributions.

In some aspects, the bootstrap resampling procedure comprises at least 1,000 iterations. For each iteration, an equal number of samples (n=group size) may be drawn with replacement from each group, and the mean difference between groups is calculated for the metric of interest. This yields a bootstrap distribution of mean differences. In some aspects, empirical confidence intervals are defined by the 2.5th and 97.5th percentiles of the bootstrap distribution, representing a 95% confidence interval. A difference is considered statistically significant if zero is not contained within this interval.

In some aspects, the bootstrap resampling procedure may be applied to baseline metrics (e.g., mPSDbrady, mPSDnormo, and mPSDtachy) to verify that there are no pre-existing differences between EGG baseline (pre-feeding) power offsets between comparison groups. In some aspects, the bootstrap resampling procedure may be applied to mPR metrics (e.g., mPRDur/Pre and mPRPost/Pre values within the bradygastria, normogastria, and tachygastria bands) to assess group differences in feeding-related gastric activity. This approach ensures that statistical conclusions are data-driven and distribution-free, strengthening the validity of observed group differences.

### C. Computer Implemented Methods

In some aspects, the current disclosure also encompasses a computer-implemented method for predicting and/or diagnosing feeding intolerance (FI) using pre- and during feeding electrogastrography (EGG) signal data to obtain mean power ratios mPRdur/pre (mean power ratio) from power spectral density (PSD) obtained from during and pre-feeding EGG signals.

In some aspects, certain processes and methods described herein (e.g., obtaining data, quantifying, normalizing, range setting, adjusting, categorizing, clustering, counting, setting thresholds, profiles, and data analysis and integration) often cannot be performed without a computer, processor, software, module or other apparatus. Methods described herein may typically comprise computer-implemented methods, and one or more portions of a method sometimes are performed by one or more processors. Aspects pertaining to methods described herein generally are applicable to the same or related processes implemented by instructions in systems, apparatus and computer program products described herein. In some aspects, processes and methods described herein (e.g., quantifying, normalizing, range setting, adjusting, categorizing, clustering, counting profiles, setting thresholds and data analysis and integration) are performed by automated or semi-automated methods. In some aspects, an automated method is embodied in software, modules, processors, peripherals and/or an apparatus comprising the like, that determine sequence reads, counts, profiles, normalizations, comparisons, range setting, categorization, adjustments, plotting, outcomes, transformations, identifications, clustering, counting profiles, setting thresholds and data analysis and integration. As used herein, software refers to computer readable program instructions that, when executed by a processor, perform computer operations, as described herein. In some aspects, data or data sets can be characterized by one or more features or variables. In some aspects, data or data sets can be feature independent. In certain aspects, data or data sets can be organized into a matrix having two or more dimensions based on one or more features or variables. Data organized into matrices can be organized using any suitable features or variables. Exemplary software for use with the disclosed method include, but are not limited to:

MATLAB—Widely used for signal processing, MATLAB provides toolboxes such as the Signal Processing Toolbox and Wavelet Toolbox, which can analyze EGG data through spectral analysis, filtering, and feature extraction.

LabChart (ADInstruments)—A commercial software suite that supports EGG data acquisition and analysis, including spectral analysis and slow-wave detection.

BioPac AcqKnowledge—Compatible with BioPac hardware, this software offers real-time EGG signal processing, including noise reduction and power spectral density analysis.

EEGLAB (MATLAB Toolbox)—Though primarily designed for EEG, EEGLAB can process EGG data with features like independent component analysis (ICA) for artifact removal.

Python (SciPy, NumPy, MNE-Python)—Open-source libraries such as SciPy and NumPy enable EGG data processing, while MNE-Python, designed for electrophysiological signals, can be adapted for EGG analysis.

Custom-built software (C++, Python, or MATLAB-based)—Many researchers develop proprietary software tailored to specific EGG processing needs, incorporating advanced filtering, spectral estimation, and machine learning algorithms.

OriginPro—A statistical and signal analysis tool that allows for EGG data visualization, filtering, and power spectrum analysis.

Spike2 (CED)—Used for electrophysiological data analysis, Spike2 includes features for time-domain and frequency-domain analysis of EGG signals.

Also, disclosed herein are systems for implementing the disclosed method, comprising the steps of: (a) providing pre- and during feeding electrogastrography (EGG) signal data from at least one feed for the subject; (b) obtaining mean power ratios mPRdur/pre (mean power ratio) from power spectral density (PSD) obtained from during and pre-feeding EGG signals; and (c) predicting/diagnosing feeding intolerance in the subject by determining the mPRdur/pre.

A system typically comprises one or more apparatus. Each apparatus comprises one or more of memory, one or more processors, and instructions. Where a system includes two or more apparatus, some or all of the apparatus may be located at the same location, some or all of the apparatus may be located at different locations, all of the apparatus may be located at one location and/or all of the apparatus may be located at different locations. Where a system includes two or more apparatus, some or all of the apparatus may be located at the same location as a user, some or all of the apparatus may be located at a location different than a user, all of the apparatus may be located at the same location as the user, and/or all of the apparatus may be located at one or more locations different than the user.

In an aspect, the system may comprise one or more computing apparatus or one or more measuring apparatus (for example, an EGG apparatus and a computer) or any combination thereof. The EGG apparatus may be configured to receive data and the computing apparatus is configured to process the data, and further generate profiles, compare profiles and/or identify patient types. In some aspects, output from an apparatus may serve as data that can be input via an input device to one or more computing apparatus. Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In certain aspects, EGG data may serve as data that can be input via an input device. In certain aspects, data is generated by an in silico process and serves as data that can be input via an input device. The term “in silico” refers to research and experiments performed using a computer. In silico processes include, but are not limited to, processing and transforming EGG signal data received from patients into usable diagnostic information.

Systems addressed herein may comprise general components of computer systems, such as, for example, network servers, laptop systems, desktop systems, handheld systems, personal digital assistants, computing kiosks, and the like.

A system and/or apparatus may include software useful for performing a process described herein, and software can include one or more modules for performing such processes (e.g., graphing modules, signal transforming modules, logic processing module, data display organization module). The term “software” refers to computer readable program instructions that, when executed by a computer, perform computer operations. Instructions executable by the one or more processors sometimes are provided as executable code, that when executed, can cause one or more processors to implement a method described herein. A module described herein can exist as software, and instructions (e.g., processes, routines, subroutines) embodied in the software can be implemented or performed by a processor. For example, a module (e.g., a software module) can be a part of a program that performs a particular process or task. The term “module” refers to a self-contained functional unit that can be used in a larger apparatus or software system. A module can comprise a set of instructions for carrying out a function of the module. A module can transform data and/or information. Data and/or information can be in a suitable form. For example, data and/or information can be digital or analogue. In some cases, data and/or information can be packets, bytes, characters, or bits. In some aspects, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, elevations, ranges, thresholds, signals, displays, representations, or transformations thereof.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash drives, RAM, floppy discs, the like, and other such media on which the program instructions can be recorded. In online implementation, a server and web site maintained by an organization can be configured to provide software downloads to remote users, or remote users may access a remote system maintained by an organization to remotely access software.

Software can include one or more algorithms in certain aspects. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some aspects, can be used in a deterministic or probabilistic/predictive approach. An algorithm can be implemented in a computing environment by use of a suitable programming language, non-limiting examples of which are C, C++, Java, Perl, Python, Fortran, and the like. In some aspects, an algorithm can be configured or modified to include margin of errors, statistical analysis, statistical significance, and/or comparison to other information or data sets (e.g., applicable when using a neural net or clustering algorithm).

In certain aspects, several algorithms may be implemented for use in software. These algorithms can be trained with raw data in some aspects. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some aspects. An algorithm with the highest sensitivity and/or specificity may be identified and utilized, in certain aspects.

In certain aspects, simulated (or simulation) data can aid data processing, for example, by training an algorithm or testing an algorithm. In some aspects, simulated data includes hypothetical various samplings of different groupings. Simulated data may be based on what might be expected from a real population or may be skewed to test an algorithm and/or to assign a correct classification. Simulated data also is referred to herein as “virtual” data. Simulations can be performed by a computer program in certain aspects. One possible step in using a simulated data set is to evaluate the confidence of an identified results, e.g., how well a random sampling matches or best represents the original data. One approach is to calculate a probability value (p-value), which estimates the probability of a random sample having better score than the selected samples. In some aspects, an empirical model may be assessed, in which it is assumed that at least one sample matches a reference sample (with or without resolved variations). In some aspects, another distribution, such as a Poisson distribution for example, can be used to define the probability distribution.

Sometimes peripherals and components assist an apparatus in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., iPad®, tablets), touch screens, smart phones, mobile phones, USB I/O devices, USB mass storage devices, keyboards, a computer mouse, digital pens, modems, hard drives, jump drives, flash drives, a processor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

In some aspects, also disclosed are computer program products, such as, for example, a computer program product comprising a computer usable medium having a computer readable program code embodied therein, the computer readable program code adapted to be executed to implement a method comprising: (a) providing pre- and during feeding electrogastrography (EGG) signal data from at least one feeds for the subject; (b) obtaining mean power ratios mPRdur/pre (mean power ratio) from power spectral density (PSD) obtained from during and pre-feeding EGG signals; and (c) predicting/diagnosing feeding intolerance in the subject based on the mPRdur/pre ratio.

### III. Gut Microbiota-Derived Metabolites Assays to Determine Feed Intolerance (FI)

In some aspects, the current disclosure also encompasses methods for diagnosing/predicting FI in a subject, comprising: (a) obtaining or having obtained biological sample from the preterm infant; (b) determining a level of one or more microbiota-derived metabolites in the biological sample; (c) comparing the level of the one or more microbiota derived metabolite to a standard level; (d) diagnosing/predicting FI if the level of the one or more microbiota derived metabolite is different from a reference level. In some aspects, the current disclosure also encompasses method distinguishing between the type of FI, for example DFI, or PFI in a subject in need thereof, using steps (a)-(d). In some aspects, the disclosure also encompasses methods of treating a FI in a subject if the diagnoses is positive. In some aspects, the current disclosure also encompasses combining the one or more methods of predicting/diagnosing FI, for example an EGG based method and a biomarker based method as disclosed in this section.

In some aspects, the identification and use of gut microbiota-derived metabolite biomarkers may be achieved by analyzing biological samples obtained from the subject using techniques known in the art. Metabolic biomarkers can be identified through techniques such as mass spectrometry, nuclear magnetic resonance (NMR) spectroscopy, and high-performance liquid chromatography (HPLC). These biomarkers include metabolites such as glucose, lipids, amino acids, vitamins and other small molecules that provide insight into cellular processes and disease states. In some aspects, the biological sample is fecal matter.

To determine clinically relevant metabolic biomarkers, subject samples can be analyzed to detect consistent metabolic alterations associated with feeding intolerance as described in the example below. Statistical analysis, including machine learning techniques, can be applied to large datasets to identify significant correlations between metabolic profiles and disease progression. As provided in the example, once identified, these biomarkers can be validated through independent cohorts and functional assays.

Biomarker thresholds (e.g., reference levels) may be evaluated using the median levels in a population, or by considering quartiles or tertiles as reference points. Various statistical methods, can be employed to determine the most suitable cut-off, where the variation in treatment response between biomarker-high and biomarker-low populations is most pronounced. Additional datasets and descriptive statistics of biomarker distribution can also aid in selecting clinically relevant thresholds.

In some aspects, the biological sample is fecal matter obtained from a pre-term infant and the one or more microbiota-derived metabolites are alpha-tocopherol and/or gamma-tocopherol. In some aspects, the level of alpha-tocopherol and/or gamma-tocopherol is at least about 5% to about 50% or more, higher in subjects with PFI compared to subjects with NFI or DFI. In some aspects, the level of alpha- and/or gamma-tocopherol in the fecal sample derived from pre-term infants with PFI is at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more that a reference level, wherein the reference level corresponds to a subject, or a population of subject with NFI or DFI.

### IV. Method of Diagnosing and Treatment

In some aspects, the disclosed method can be used to predict, diagnose and/or distinguish between various types of FI, for example DFI and PFI. In some aspects, the disclosed method can be further used to inform treatment decisions based on the diagnosed form of FI, for example DFI and PFI.

In some aspects, disclosed herein is a method of treating a subject in need thereof for a feeding intolerance (FI) comprising determining a mean power ratio mPRdur/pre using the steps of: (a) providing pre- and during feeding electrogastrography (EGG) signal data from at least one feed for the subject; and (b) obtaining mean power ratios mPRdur/pre from power spectral density (PSD) obtained from during and pre-feeding EGG signals, and administering a treatment to the subject, wherein the subject has been determined to have the mPRdur/pre ratio of less than or equal to one. In some aspects, disclosed herein is a method of treating a subject in need thereof for a FI, comprising: (a) determining the levels of one or more microbiome derived metabolites in a biological sample obtained from the subject; (b) comparing the levels of the one or more microbiome derived metabolites in the biological sample with a reference level; (c) administering a treatment when the subject is determined to have changed levels of the one or more microbiome derived metabolites compared to the reference level.

In some aspects, the subject is a mammal, for example primates, ovine, canine, feline, horse, rodents, or bovines. In some aspects, the subject is a mammalian infant. In some aspects, the subject is a human. In some aspects, the subject is a human infant. In some aspects, the subject is a pre-term baby. In some aspects, the infant is born prior to 37 weeks of gestation. In some aspects, the infant is born before, at least, equal to, or at most 28, 29, 30, 31, 32, 33, 34, 35, 36, or 37 weeks of gestation. In some aspects, the subject is a human infant born after 37 weeks, 38 weeks, 39 weeks, 40 weeks of gestation. In some aspects, the subject is a human infant less than, equal to, or more than 1 week, 2 week, 3 weeks, 4 weeks, 1, 2, 3, 4, 5, 6, 7, 8, 9, 10, 11, 12 or more months in age.

In some aspects, the method of prediction and/or diagnosis comprises: providing pre- and during feeding electrogastrography (EGG) signal data from at least one feed for the subject; obtaining mean power ratios mPRdur/pre from power spectral density (PSD) obtained from during and pre-feeding EGG signals; comparing the mean power ratio to a baseline value, determine either from data from the same subject, data from one or more different subjects, or a population average. In some aspects, the subject is predicted and/or diagnosed with a FI if the subject exhibits a mean power ratios mPRdur/pre of less than or equal to one. In some aspects, the subject is predicted or diagnosed to be free of a FI (NFI) if the mean power ratios mPRdur/pre is greater than 1.

In some aspects, the current disclosure also encompasses a method of treating a subject diagnosed with a FI using the method disclosed herein. In some aspects, the treatment may comprise one or more of minimal enteral nutrition (MEN), slow advancement of feeds, use of human milk, fortified feeds, parenteral nutrition, hydrolyzed or elemental formulas, prokinetic agents, upright or lateral positioning, probiotics, prebiotics, nutritional supplements, avoidance of unnecessary antibiotics, gastric residual monitoring, continuous vs. bolus feeding adjustments, abdominal massage, non-nutritive sucking, thickened feeds for reflux, acid suppressants in severe gastroesophageal reflux disease (GERD), bowel rest (NPO), intravenous fluids, broad-spectrum antibiotics, surgical intervention for necrosis or obstruction, or any combination thereof.

### EXAMPLES

### Example 1

Feeding intolerance (FI) is a common complication in preterm infants and is reported in up to 75% of those with very low birth weight. Clinically, FI is characterized by an inability to tolerate enteral feedings and is typically inferred from non-specific signs such as gastric residual volumes exceeding 50% of the previous feed, abdominal distention, emesis, or interruptions in the planned feeding schedule. These features are widely cited across studies as defining attributes of FI; however, their interpretation remains largely subjective and varies considerably between clinicians. Moreover, FI can arise from two fundamentally different mechanisms: developmental feeding intolerance (DFI), which reflects transient gastrointestinal immaturity and dysmotility, and pathological feeding intolerance (PFI), which results from conditions such as ileus due to sepsis, necrotizing enterocolitis (NEC), spontaneous intestinal perforation, or bowel obstruction. Because the same nonspecific signs can be observed in both scenarios, DFI is frequently misinterpreted as PFI, leading to unnecessary interruption or delay of feeds in infants who may simply require maturation rather than intervention. Current bedside assessments rely heavily on observation rather than direct measurement of gastric activity and, as a result, provide limited insight into the underlying physiological state. This underscores the need for an objective and non-invasive method to quantify gastric function and, ultimately, to distinguish a benign developmental process from true pathology.

Electrogastrography (EGG) is a noninvasive technique that records gastric myoelectrical activity from abdominal surface electrodes, primarily capturing the slow-wave component known as the gastric rhythm (GR). These signals are weak and often contaminated by respiration, motion, and nearby organ activity, making spectral analysis the standard approach for extracting meaningful information. Power spectral density (PSD) enables quantification of gastric rhythms across frequency bands: bradygastria (brady: 0.5<GR<2 cpm), normogastria (normo: 2<GR<4 cpm), and tachygastria (tachy: 4<GR<9 cpm). Since its introduction in the 1920s, EGG has been applied in adults, children, and preterm infants under both physiological and pathological conditions, with standardized parameters such as dominant frequency, dominant power, power ratio (postprandial vs. preprandial baseline), and rhythm stability (% time in normogastria) widely used to characterize gastric activity. Together with additional indices like instability coefficients and power distribution, these measures form the foundation of EGG interpretation in both clinical and research settings.

In adults, individuals with functional dyspepsia exhibited increased tachygastria and reduced normogastria compared to healthy controls, particularly in the fasting state. Adults with gastroparesis and delayed gastric emptying showed both diminished normogastria and a blunted postpran-dial rise in dominant power. Importantly, postprandial EGG abnormalities predicted delayed emptying with more than 75% accuracy, supporting EGG as a noninvasive marker of motility impairment. Nevertheless, EGG has not been adopted in routine clinical practice, largely due to persistent challenges with signal quality, diagnostic specificity, standardization, and clinical validation.

Despite numerous studies on infant EEG, no prior work, has specifically applied EEG to detect feeding intolerance (FI) in pre-term newborns. Infants with FI are expected to exhibit abnormal gastric motility and, consequently, altered PSD ratios in response to feeding compared with infants with no FI (NFI). Accordingly, we hypothesized that EGG-derived PSD ratios across pre-, during-, and post-feeding periods in the brady, normo, and tachy bands can distinguish infants with FI from NFI based on their gastric myoelectrical responses to feeding. The results from this study suggest that EGG may provide a feasible and unique avenue for clinical application.

In this study, PSDs were computed for each newborn during pre-, during-, and post-feeding periods, and PSD ratios were derived to assess feeding-related changes. After confirming comparable baseline gastric activity between FI and NFI groups using bootstrap analysis, frequency-specific PSD power ratios were calculated and compared, enabling discrimination between infants with and without feeding intolerance. The results revealed significantly lower PSD ratios between during- and pre-feeding periods in the brady and tachy bands in FI infants, supporting the hypothesis and indicating an attenuated gastric response to feeding in neonates with FI.

### Example 2: Methods Used in the Study

This longitudinal, prospective cohort study was conducted in the neonatal intensive care units (NICUs) at Parkland Health and Hospital System and Children's Health in Dallas, Texas, between 2017 and 2022. The study protocol was approved by the Institutional Review Board at UT Southwestern Medical Center. Parental informed consent was obtained before enrollment. Participants underwent weekly EGG monitoring beginning within the first 14 postnatal days and continuing until 40 weeks postmenstrual age (PMA), hospital discharge, or death, whichever occurred first. However, the primary focus of this study was on measurements obtained during the first postnatal week, or the second week when first-week data were unavailable.

A total of 100 infants were enrolled (see Table 1). Of these, 84 infants (79 preterm and 5 term) met the study criteria and were included in the analysis. Participants were preterm infants <34 weeks' gestational age (GA) and term infants ≥37 weeks' GA at birth. Term infants were included as a study reference. Exclusion criteria were known congenital or chromosomal disorders, significant clinical instability, major skin abnormalities precluding electrode placement, nil per os (NPO) status or continuous feeding at the time of measurement, and absence of an early EGG recording during week 1 and week 2 of life (see Table 2).

Infants were divided into two cohorts based on objective criteria, instead of non-specific clinical measures. The no feeding intolerance (NFI) group (n=75) met both criteria of sustained feeding tolerance: 1) achieved full enteral feeds (≥120 mL/Kg/day) sustained for ≥5 days without parenteral (intravenous) nutrition and 2) had a growth velocity of ≥10 g/Kg/day from the 1st day of full enteral feeds until 34 weeks+6 days postmenstrual age. The feeding intolerance (FI) group (n=9) met 0-1 criterium and/or developed gastrointestinal pathology (ileus due to sepsis, necrotizing enterocolitis Stage ≥Ila, spontaneous intestinal perforation, or bowel obstruction). The FI group consisted of developmental FI (DFI, n=5) and pathological FI (PFI, n=4), though the primary analyses focused on FI versus NFI.

TABLE 1
Demographics of Comparison Groups
(n = 84) used for Examples 1 and 3.

| Number (n) of patients | NFI (n = 75) | | DFI (n = 5) | | PFI (n = 4) | |
|---|---|---|---|---|---|---|
| Gestational Age (weeks) | 29 | | 30 | | 27 | |
| | [24-39] | | [26-33] | | [25-28] | |
| Birthweight (g) | 1391 | | 1350 | | 895 | |
| | [450-4245] | | [810-2000] | | [710-1150] | |
| Sex | | | | | | |
| Female | 41 | [55%] | 3 | [60%] | 2 | [50%] |
| Male | 34 | [45%] | 2 | [40%] | 2 | [50%] |
| Race/Ethnicity | | | | | | |
| Black Non-Hispanic/Latino | 20 | [27%] | 3 | [60%] | 3 | [75%] |
| White Non-Hispanic/Latino | 2 | [3%] | 0 | [0] | 0 | [0%] |
| Hispanic/Latino | 50 | [67%] | 2 | [40%] | 1 | [25%] |
| Asian | 1 | [1%] | 0 | [0] | 0 | [0] |
| Unknown/Not Reported | 2 | [3%] | 0 | [0] | 0 | [0] |
| Feeding Type | | | | | | |
| Mother's Own Milk (MoM) | 45 | [60%] | 3 | [60%] | 4 | [100%] |
| Donor Human Milk (DHM) | 11 | [15%] | 1 | [20%] | 0 | [0%] |
| Formula | 18 | [25%] | 1 | [20%] | 0 | [0%] |
| Mixture of MoM and DHM | 0 | [0%] | 0 | [0%] | 0 | [0%] |
| Mixture of MoM and | 1 | [1%] | 0 | [0%] | 0 | [0%] |
| Formula | | | | | | |
| Feeding Volume (mL/Kg) | 11.7 | | 9.5 | | 13.1 | |
| | [1.6-20.4] | | [2.5-15.4] | | [9.1-17.2] | |
| Age of First Feed (days) | 2 | | 2 | | 2 | |
| | [1-4] | | [1-3] | | [2-3] | |
| Days of Hospitalization | 68 | | 62 | | 90 | |
| | [3-197] | | [15-151] | | [39-134] | |
| Days on Parenteral Nutrition | 11 | | 10 | | 60 | |
| | [0-35] | | [0-17] | | [32-103] | |
| Central Line Days | 10 | | 8 | | 40 | |
| | [0-38] | | [0-13] | | [8-105] | |
| Days to Full Feeds | 9 | | 8 | | 20 | |
| | [2-35] | | [6-9] | | [6-34] | |
| Days to Sustained Feeding | 10 | | 8 | | 25 | |
| Tolerance (SFT)* | [4-34] | | [6-10] | | [7-53] | |
| Number of Times NPO | 2 | | 1 | | 6 | |
| | [0-10] | | [0-3] | | [3-14] | |
| Number of Days NPO | 2 | | 1 | | 16 | |
| | [0-11] | | [0-3] | | [9-29] | |
| Growth Velocity, g/Kg/day | 15 | | 9 | | 5 | |
| | [10-29] | | [8-10] | | [−11-14] | |
| NEC Stage ≥ IIa | | | | | | |
| Medical | 0 | [0%] | 0 | [0%] | 1 | [25%] |
| Surgical or Death | 0 | [0%] | 0 | [0%] | 2 | [50%] |
| Bowel Obstruction (Volvulus) | 0 | [0%] | 0 | [0%] | 1 | [25%] |
| Death | 0 | [0%] | 0 | [0%] | 1 | [25%] |

Mean (range) is presented for continuous variables and n (%) for categorical variables. Growth velocity from 1st day of sustained feeding tolerance to 34 w 6 d postmenstrual age, DFI = developmental feeding intolerance, PFI = pathologic feeding intolerance, NFI = no feeding intolerance, SFT = sustained feeding tolerance (ability to achieve ≥120 ml/kg/day of full enteral feeds for ≥5 days without parenteral nutrition).

TABLE 2
Inclusion and Exclusion Criteria

| Inclusion Criteria | Exclusion Criteria |
|---|---|
| Infants born <34 or ≥37 weeks' gestation | Known congenital or chromosomal disorders |
| Admission to the neonatal intensive care unit | Significant clinical instability |
| Receiving enteral feeds | Major skin abnormalities |

Subsequently, the neonates were stratified based on gut health, categorized as either NFI (No Feeding Intolerance), 83 out of 100 infants, or FI (Feeding Intolerance), 17 out of 100 infants.

To ensure data integrity, the first-week measurement was employed in the study. In cases where this data was unavailable, the second-week measurement was considered. Infants lacking both measurements were excluded from the cohort (16 infants). Ultimately, a total of 84 babies were included in the study, comprising 74 NFI and 9 FI infants.

### Setup and Experimental Paradigm

### Electrogastrography (EGG)

Neonatal EGG electrodes were positioned on the abdominal skin of each neonate following validated protocols. Due to the limited abdominal size of neonates, the setup was confined to three electrodes (FIGS. 1A and 1B). Data acquisition was performed using the BIOPAC® MP36R System by BIOPAC® Systems, Inc., Goleta, CA.

### ElectroGastroGram (EGG) Measurements

EGG recordings were conducted on a weekly basis for a duration of six hours to encompass two feeding sessions per infant. Each recording session spanned approximately six hours, commencing 30 minutes before the first feeding and concluding 30-150 minutes after the second feeding. Thus, each recording comprised two pre-feeding, two during-feeding, and two post-feeding segments, each lasting approximately 30 minutes.

### Data Processing

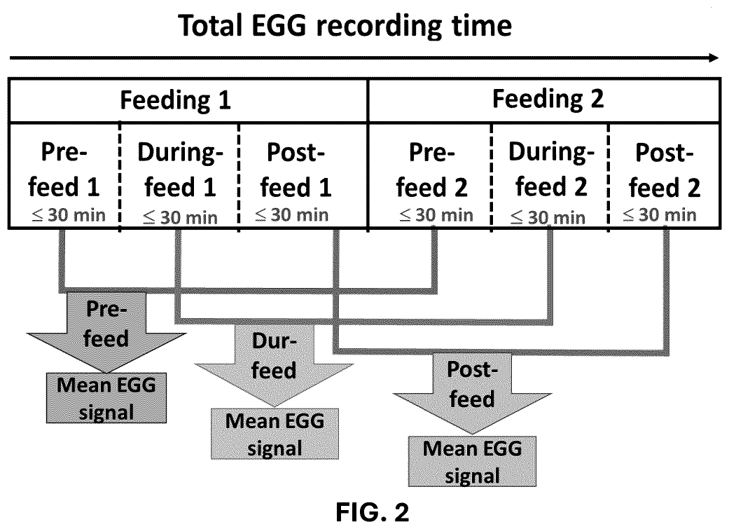

EGG data preprocessing: The initial step involved collecting raw EGG data at a sampling frequency of 2000 Hz, followed by preprocessing using MATLAB (Mathworks®, Natick, Massachusetts). This preprocessing procedure included several stages: (1) downsampling the data to 500 Hz; (2) applying a third-order polynomial fit using MATLAB to detrend the data and capture temporal trends in the downsampled time series; (3) subtracting the fitted trend from the downsampled data; and (4) filtering the detrended time series using a low-pass filter at 1 Hz in MATLAB to mitigate potential filtering-induced phase shifts. A flowchart of the processing procedure is shown in FIG. 2 and a detailed discussion of the different steps is provided in the following.

The above method was further modified as described herein. Raw EGG recordings were processed in MATLAB (MathWorks®, Natick, MA) following a standardized pipeline. The data were first down sampled from 2000 Hz to 500 Hz to reduce computational load while preserving the frequency content of interest. A third-order polynomial fit was applied to obtain and then remove signal drift from the down sampled signal. The detrended signal was then low-pass filtered at 0.37 Hz (~22.2 cpm) using a zero-phase second-order Butterworth filter to attenuate high-frequency noise while avoiding phase distortion. For each of the six feeding segments, the PSD was computed over the gastric frequency band (0.5-9 cpm) using Welch's method. Processing was performed on signals down-sampled at 500 Hz and low-pass filtered at 0.37 Hz, with a 4-minute window, 2-minute overlap, and a resulting frequency resolution of 0.004 Hz (=0.24 cpm).

Selections of sub-feeding periods: Utilizing the timestamps corresponding to the initiation and completion of feeding1 and feeding2, the pre-processed dataset was partitioned into six distinct segments: pre-feeding 1, during-feeding 1, post-feeding 1, pre-feeding 2, during-feeding 2, and post-feeding 2. This segmentation facilitated a comprehensive analysis of gastric activity across different phases of feeding for each participant.

### Steps to Obtain EGG Power Spectral Density (PSD) and Spectral Means at Three Gastric Frequency Bands:

Step 1: The power spectral density (PSD) across the entire gastric frequency band (0.5-9 cpm) during each of the six feeding periods (i.e., Pre-feed 1, During-feed 1, Post-feed 1, Pre-feed 2, During-feed 2, and Post-feed 2) was computed using MATLAB. This analysis was conducted with a down-sampled frequency of 500 Hz, employing a 4-minute window and 2-minute overlap for each neonate. The utilization of a 4-minute window resulted in a frequency resolution of 0.004 Hz (or 4 mHz) in the PSD calculation, particularly between 0 to 1 Hz.

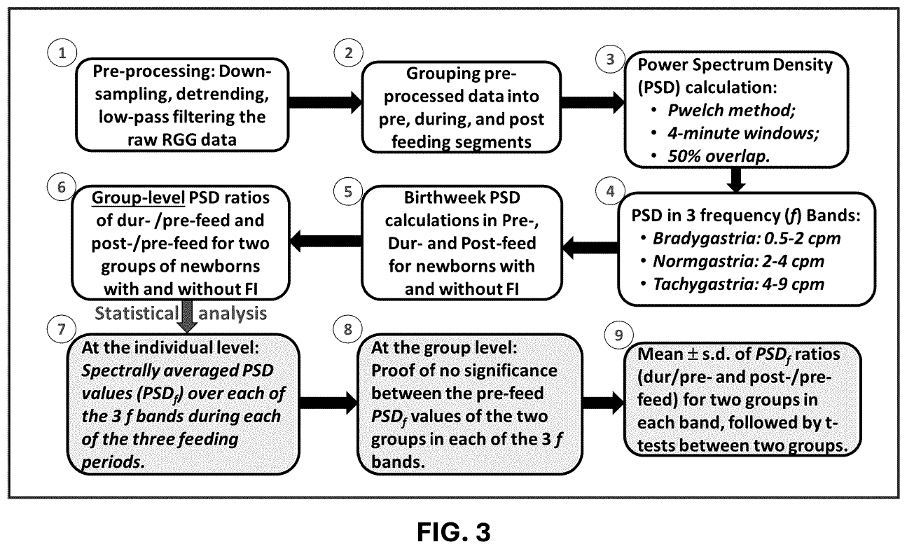

Step 2: Quantification of mean PSD values over three sub-feeding periods by averaging them between Pre-feed 1 and Pre-feed 2, During-feed 1 and During-feed 2, and Post-feed 1 and Post-feed 2, if two feeds are available (FIG. 3).

Step 3: Following the acquisition of the three power spectral density (PSD) curves representing the pre-, during-, and post-feeding phases for each infant, the mean PSD curves for each phase was calculated by averaging the PSD data from 75 infants classified as NFI and separately for the 9 infants categorized as FI. This process resulted in three distinct mean PSD curves for both the NFI and FI groups.

Step 4: After obtaining the power spectral density (PSD) values for the during-feeding and post-feeding periods for each infant, two PSD ratio curves were computed. These were derived by dividing the PSD values during feeding and after feeding by pre-feeding phase. This process yielded two distinct PSD ratio curves, representing the relative changes in gastric activity during and after feeding compared to the pre-feeding baseline for each baby. Subsequently, the average of these PSD ratio curves (Dur/Pre and Post/Pre) within each group of infants categorized as either NFI or FI were calculated.

Step 5: Statistical analysis: Initially, the mean power spectral density (PSD) value was computed within specific frequency bands, namely Bradygastria (0.5-2 cpm), Normogastria (2-4 cpm), and Tachygastria (4-9 cpm), for all infants across three feeding periods: pre-, during-, and post-feeding. This resulted in the determination of three distinct values—mPSDbrady, mPSDnormo, and mPSDtachy—for each baby at each feeding phase.

Step 6: Further analysis was conducted by averaging the three values of mPSDbrady, mPSDnormo, and mPSDtachy for the pre-feeding period within each group of infants categorized as either NFI or FI. Subsequently, independent t-tests were performed to compare the mPSD values between the NFI and FI groups for each frequency band (mPSDbrady, mPSDnormo, and mPSDtachy).

Step 7: Initially, the ratio values of Dur/Pre and Post/Pre was computed for each baby at each frequency band, resulting in a total of six values for each infant. Subsequently, the average of these six ratio values was determined within each group of infants classified as either NFI or FI. Following this, t-tests were conducted between each pair of NFI and FI groups, comparing the average ratio values for Dur/Pre and Post/Pre across all frequency bands.

### Statistical Analysis Using Bootstrap Resampling

Since PSD power comparisons were based on ratios of RDur/Pre or RPost/Pre between the two groups, it was necessary to verify that there were no pre-existing differences between EGG baseline (pre-feeding) power offsets of FI and NFI infant groups. To facilitate statistical comparison of baseline gastric activity between the two groups, the pre-feeding PSD curves were used as the reference signals for respective groups. At the individual level, three mean PSD (mPSD) values were then obtained by averaging the PSD of pre-feeding within the three given gastric frequency bands: bradygastria, normogastria, and tachygastria, yielding mPSDbrady, mPSDnormo, and mPSDtachy.

At the group level, to compare baseline gastric activity between the NFI (n=75) and FI (n=9) groups, a nonparametric bootstrap resampling procedure with replacement was used to account for the unequal sample sizes. This procedure was applied separately to each of the three baseline metrics (i.e., mPSDbrady, mPSDnormo, and mPSDtachy). For each of 1,000 iterations, an equal number of samples (n=group size) were drawn with replacement from each group, and the mean difference between groups was calculated for the metric of interest. This yielded a bootstrap distribution of mean differences, from which the 2.5th and 97.5th percentiles were taken as the empirical 95% confidence interval. A difference was considered statistically significant if zero was not contained within this interval.

Calculations and Comparisons of Mean Power Ratio (mPR) Metrics

After confirming no baseline differences in EGG between the two groups, mPR metrics were calculated for each infant by averaging RDur/Pre and RPost/Pre values within the bradygastria, normogastria, and tachygastria bands. This yielded six metrics per infant: mPRDur/Pre_b, mPRDur/Pre_n, mPRDur/Pre_t, mPRPost/Pre_b, mPRPost/Pre_n, and mPRPost/Pre_t for both during- and post-feeding periods. To assess group differences, the same bootstrap resampling procedure was applied to each metric, with 1,000 iterations used to generate the distribution of mean differences between the NFI and FI groups. Empirical 95% confidence intervals were defined by the 2.5th and 97.5th percentiles of these distributions, and statistical significance was determined when the interval excluded zero. Another methodological innovation in this study is the use of a non-parametric bootstrap resampling technique to compare groups with unequal sample sizes. Bootstrapping allows the estimation of empirical confidence intervals under data imbalance. This approach ensures that statistical conclusions are data-driven and distribution-free, strengthening the validity of the observed group differences and demonstrating a rigorous analytical framework applicable to future neonatal EGG.

### Example 2: Results

Study cohort. A total of 84 infants were included in the study, with 75 classified as NFI and 9 as FI. Consent was obtained from their guardians, and the study followed Institutional Review Board approval. Procedures adhered to guidelines, and infants with certain conditions were excluded. To ensure data integrity, either first-week or second-week measurements for the infants was utilized in the analysis, based on data availability.

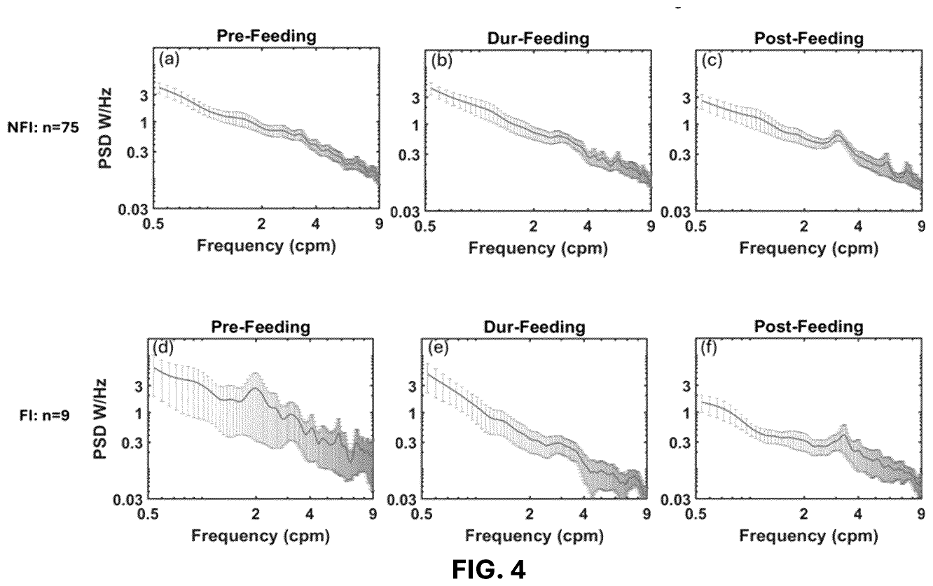

Quantification of Group-averaged mPSD over three sub-feeding periods for NFI and FI. After obtaining the power spectral density (PSD) curves representing the pre-, during-, and post-feeding phases for each infant, the mean PSD curves for each phase were computed. This involved averaging the PSD data from 75 infants classified as NFI and separately from the 9 infants categorized as FI. Consequently, three distinct mean PSD curves were generated for each of the NFI and FI groups (FIG. 4). These curves provide a visual representation of the group-averaged gastric activity across the pre-feed, during-feed, and post-feed phases.

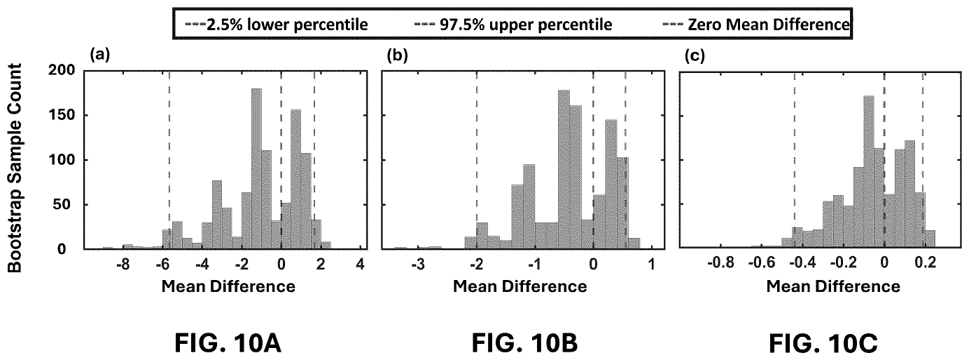

A bootstrapped method was developed to further assess baseline gastric activity. mPSD values for each frequency band—bradygastria, normogastria, and tachygastria—were calculated from the pre-feeding PSD curves of each infant. Bootstrapping with replacement was used to evaluate differences in baseline mPSD values between the NFI (n=75) and FI (n=9) groups. The bootstrapped distributions of mean differences indicated that the zero line fell within the 95% confidence intervals for all three frequency bands, suggesting no statistically significant baseline differences between two groups (see FIGS. 10A-10C).

Quantification of Group-averaged PSD Ratio Curves of Dur/Pre and Post/Pre for NFI and FI. After obtaining PSD for the during-feeding and post-feeding periods for each infant, two PSD ratio curves were calculated. These curves were generated by dividing the PSD during and after feeding by those observed during the pre-feeding phase. This process resulted in two distinct PSD ratio curves, reflecting the relative changes in gastric activity during and after feeding compared to the pre-feeding baseline for each baby.

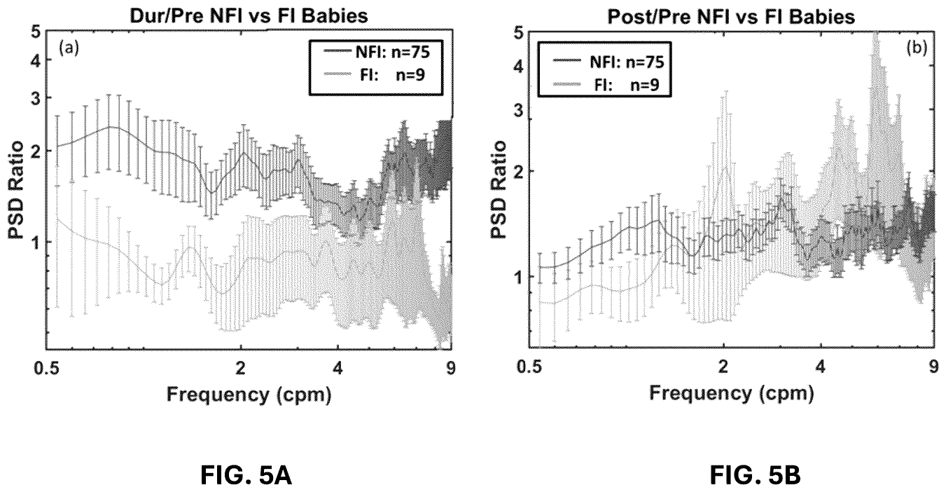

Subsequently, the average of these PSD ratio curves (Dur/Pre and Post/Pre) were computed within each group of infants classified as either NFI or FI (FIG. 5). This facilitated a comprehensive assessment of the average magnitude of change in gastric activity during and after feeding relative to the pre-feeding baseline within each group. By comparing the ratio curves of the two groups, the averaged gut response to feeding between NFI and FI groups were evaluated. Specifically, the dur/pre ratio presented in FIG. 5A served as a distinguishing factor between the two groups: the gut response to feeding for NFI groups was approximately twice the baseline, indicating increased gut activity during feeding. Conversely, the gut response for FI groups, which was approximately one, suggested a negligible response change during feeding. This distinction serves as a crucial biomarker for identifying FI. For the post/pre ratio, however, no notable difference was observed between the two groups (FIG. 5B).

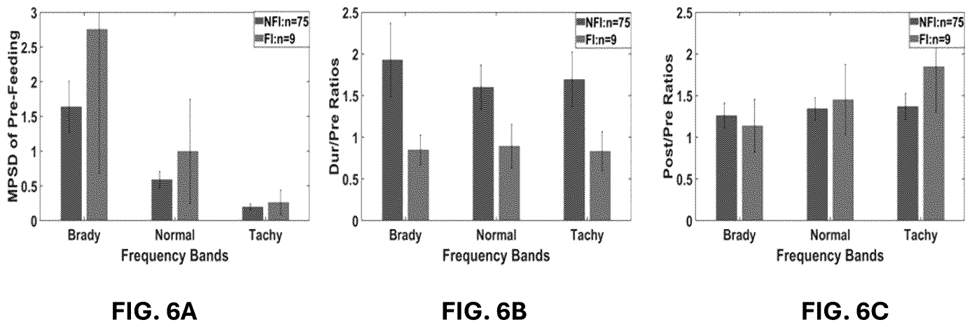

Group-averaging the three values of mPSDbrady, mPSDnormo, and mPSDtachy for NFI and FI. First, the three values of mPSDbrady, mPSDnormo, and mPSDtachy for the pre-feeding period within each infant were computed. Then, each value was averaged within the groups of infants categorized as either NFI or FI (FIG. 6A). Subsequently, t-tests were conducted to compare the mPSD values between the NFI and FI groups for each frequency band (mPSDbrady, mPSDnormo, and mPSDtachy). These results show p-values of higher than the threshold of 0.05, suggesting no statistically significant distinction in baseline gastric activity between infants with and without feeding intolerance.

Group-averaging the Dur/Pre and Post/Pre ratio values at each frequency band for NFI and FI. Initially, the ratio values of Dur/Pre and Post/Pre for each baby at each frequency band were computed, resulting in a total of six values per infant. Subsequently, the average of each ratio within each group of infants classified as either NFI or FI was obtained (FIGS. 6B and 6C). Following this, t-tests were conducted between each pair of NFI and FI groups, comparing the average ratio values for Dur/Pre and Post/Pre.

These statistical comparisons allowed evaluation of significant differences between infants with and without feeding intolerance for both Dur/Pre and Post/Pre ratios across different frequency bands. The results revealed that the p-values between NFI and FI groups for Dur/Pre ratios were approximately 0.027 and 0.038 at frequency bands of Bradygastria and Tachygastria, respectively. These findings indicate significant differences of Dur/Pre ratios between the NFI and FI groups at these frequency bands. Consequently, the ratio values of Dur/Pre at the Bradygastria and Tachygastria frequency bands serve as important biomarkers for distinguishing between NFI and FI during the first or second week after birth.

### Example 3: Gastric Activity Ratios: Bootstrapping Analysis of mPR Values

Since baseline mPSD values did not differ significantly between FI and NFI infants, relative changes in gastric activity during and after feeding were evaluated using mPR values. Accordingly, mPR ratios of mPRDur/Pre and mPRPost/Pre were calculated within each of the three gastric frequency bands for each infant. This produced six metrics per infant (mPRDur/Pre_b, mPRDur/Pre_n, and mPRDur/Pre_t by averaging PSD RDur/Pre within each respective band. In the similar way, mPRPost/Pre_b, mPRPost/Pre_n, and mPRPost/Pre_t were obtained).

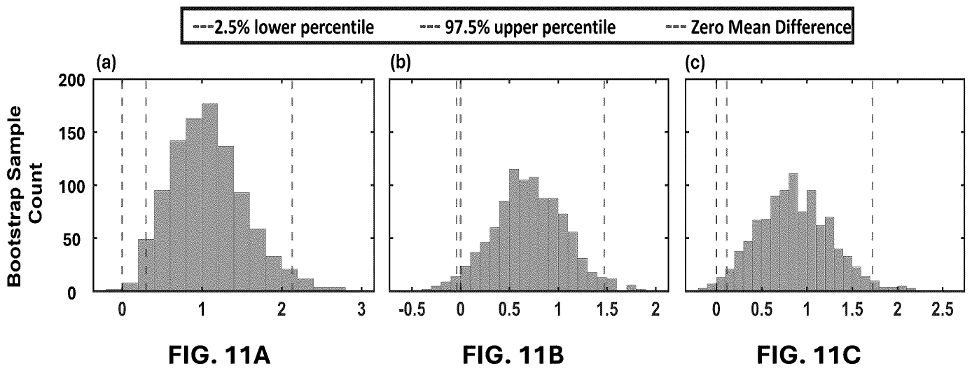

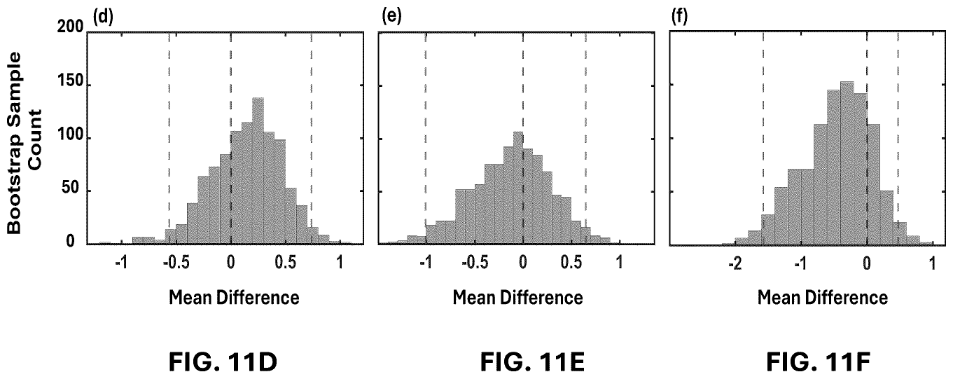

Statistical differences between the NFI and FI groups were assessed using a nonparametric bootstrap resampling approach with replacement, applied separately to each metric. Results for the three mPRDur/Pre values are shown in FIG. 11A-11C, and those for the three mPRPost/Pre values in FIG. 11D-11F. Significant differences emerged for mPRDur/Pre in the bradygastria and tachygastria bands, indicating that NFI infants exhibited stronger bradygastria and tachygastria responses during feeding. In contrast, mPRDur/Pre n values were not significantly different, suggesting that normogastria activity was similarly modulated in both groups. No significant differences were identified across any mPRPost/Pre metrics, suggesting that group-related differences in gastric responsiveness were limited to the active feeding period and did not persist afterward.

### Example 4: EGG Differences Between Developmental Feeding Intolerance (DFI), Pathologic Feeding Intolerance (PFI), and No Feeding Intolerance (NFI)

Developmental feeding intolerance (DFI) results from gastrointestinal (GI) immaturity and dysmotility, whereas pathologic feeding intolerance (PFI) is associated with ileus due to sepsis, necrotizing enterocolitis (NEC), spontaneous intestinal perforation, and bowel obstruction. Non-specific clinical signs of DFI are often mis-interpreted as PFI, leading to prolonged cessation of feeding or limitation of feeding advancement. The objective of this study was to compare differences in electrogastrography (EGG) measurements of gastric motility between infants with DFI, PFI, and no feeding intolerance (NFI).

A pilot longitudinal cohort study of infants born <34 and ≤37 weeks' gestational age who underwent weekly EGG measurements was conducted using the methods elaborated in Example 1. Table 1 provides the demographics of the comparison groups used in Example 1 and Example 3. Pre-, during-, and post-feeding EGG data from week 1-2 of life was analyzed. From raw EGG data, power spectra at frequencies 0.5-9 cycles per minute (cpm) were calculated. Mean power spectral density (mPSD) was calculated to obtain mean power ratios (mPR) between during- and pre-feeding periods (mPRdur/pre) for each comparison group. Ratios≤1 indicated dysmotility (no response to a feeding stimulus). Student's t-test was utilized to compare mPRdur/pre between comparison groups: DFI vs. NFI, PFI vs. NFI, DFI vs. PFI, and (DFI+PFI) vs. NFI.

Infants were classified into 3 comparison groups (NFI, DFI or PFI) based on objective criteria. The NFI group met BOTH criteria of sustained feeding tolerance (Table 3): 1) achieved full enteral feeds (≥120 mL/Kg/day) sustained for ≥5 days without parenteral nutrition and 2) had a growth velocity of ≥10 g/Kg/day from the 1st day of full feeds until 34 weeks plus 6 days postmenstrual age or discharge, whichever came first. The DFI group met 0-1 criterium WITHOUT GI pathology (ileus due to sepsis, NEC Stage Ella, spontaneous intestinal perforation, or bowel obstruction). The PFI group developed GI pathology.

TABLE 3
Criteria for sustained feeding tolerance
Sustained Feeding Tolerance

| Criteria 1 | Criteria 2 |
|---|---|
| Reach “full enteral feeds” of ≥120 ml/kg/day, | Growth velocity of ≥10 g/kg/day from the first |
| sustained for ≥5 days without any parenteral | day of “full enteral feeds” to 34 w + 6 d PMA or |
| nutrition. | discharge. |

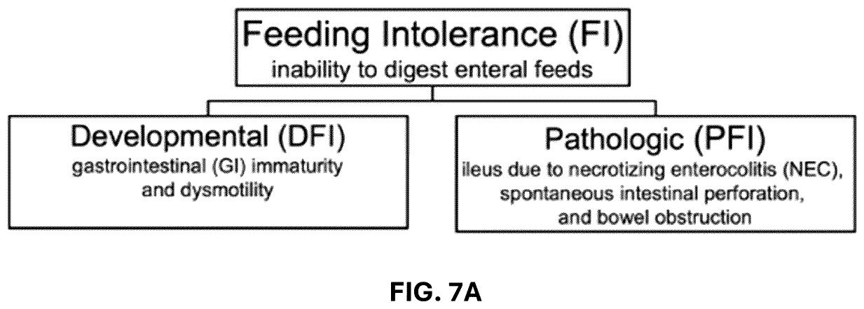

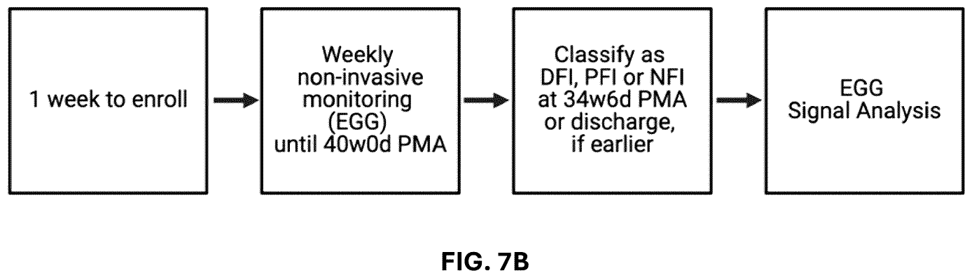

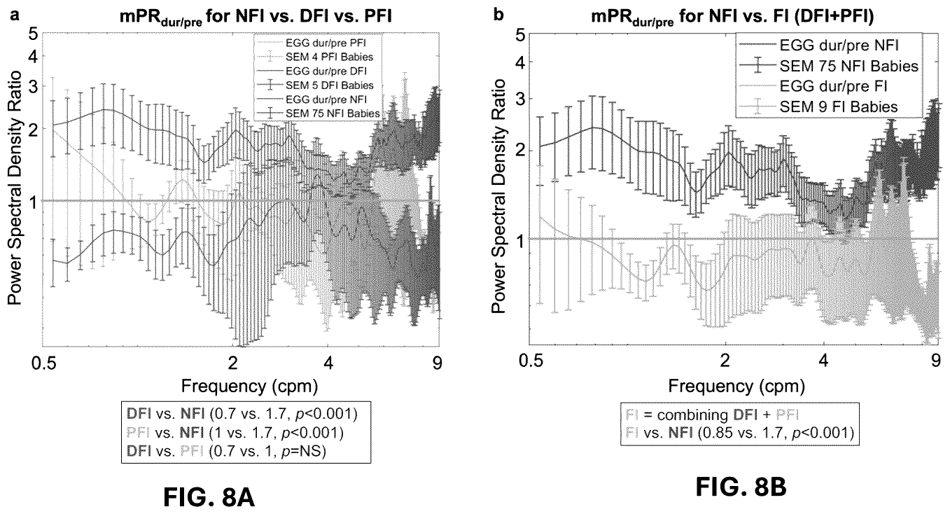

Summary of Results: 84 infants were included (75 NFI, 5 DFI, 4 PFI). PFI group included 3 NEC and 1 GI obstruction (See Table 1). A schematic of the feeding intolerance types can be found in FIG. 7A and a time line of study encounters is provided. At frequencies 0.5-9 cpm, mPRdur/pre was lower for DFI vs. NFI (0.7 vs. 1.7, p<0.001) and PFI vs. NFI (1 vs. 1.7, p<0.001), but not different for DFI vs. PFI (FIG. 8A). After combining DFI+PFI into the FI group, mPRdur/pre was lower for infants with FI than NFI (0.85 vs. 1.7, p<0.001); (FIG. 8B). For all comparisons NFI had mPRdur/pre >1 consistent with normal motility. At the low frequency band (1-2 cpm), PFI, DFI, and FI had mPRdur/pre≤1 consistent with dysmotility.

Results from this study suggest that early signs of dysmotility in preterm infants may be associated with increased risk of developing feeding intolerance. If validated, electrogastrography may be a future tool to screen infants at risk for feeding intolerance, particularly pathologic feeding intolerance such as necrotizing enterocolitis, which can lead to death or significant morbidity. Such an objective screening tool will be more helpful than the current nonspecific clinical signs and symptoms of FI that lead to prolonged cessation of feeding or limitation of feeding advancement. Current feeding interventions in response to these signs and symptoms may be unnecessary and may contribute to malnutrition and poor neurodevelopmental outcomes in children who do not have FI. If an infant were to screen positive for FI during the first 2 weeks of life, it may be possible to target therapies and interventions to this high-risk group instead of the current practice of exposing almost all infants to unnecessary interventions, such as X-ray imaging and broad spectrum antibiotics.

### Example 5: Discussion

Electrogastrography is a noninvasive method that records gastric myoelectrical activity via abdominal surface electrodes, primarily capturing the gastric slow wave that regulates contraction rhythms. Because raw signals are weak and noisy in the time domain, spectral analysis in the frequency domain is commonly used to characterize activity within three frequency bands: bradygastria (0.5-2 cpm), normogastria (2-4 cpm), and tachygastria (4-9 cpm).

Building on this foundation, the present study conducted a PSD-driven analysis of EGG signals to distinguish infants with and without FI based on their gastric myoelectrical responses to feeding. Gastric contraction rhythms were evaluated using several complementary spectral metrics—PSDs, PSD ratios, and mPR values—applied in a hierarchical manner. PSDs quantify how myoelectrical energy is distributed across frequencies, enabling assessment of activity within the brady-, normo-, and tachy-gastria bands (FIG. 4). PSD ratios were used to evaluate feeding-induced changes in gastric activity, reflecting the level of maturity or impairment in FI. For instance, a ratio≤1 in PSD RDur/Pre or RPost/Pre (FIGS. 5A and 5B) indicates little or no response to feeding, suggesting impaired gastric function. Finally, mPR metrics provided single numerical estimates across the three frequency bands, facilitating statistical comparisons between FI and NFI groups.

Group-averaged PSD ratio analysis showed that NFI infants exhibited higher RDur/Pre values (~2) in the bradygastria and tachygastria bands, reflecting the expected upregulation of gastric activity during feeding. In contrast, FI infants displayed blunted modulation of slow-wave power, with RDur/Pre values near 1, indicating absent or impaired responses. No significant group differences were observed in RPost/Pre, suggesting that alterations in gastric activity were confined only to the during feeding period. These findings were confirmed statistically (FIGS. 11A-11F): NFI infants demonstrated significantly greater bradygastria and tachygastria responses during feeding, whereas FI infants showed impaired feeding-induced myoelectrical stimulation across all frequency bands. This impairment is both statistically significant and clinically relevant, representing a potential early sensing marker for predicting feeding intolerance in preterm infants. Overall, we have proved our hypothesis that an appropriate spectral power analysis of EGG enables us to distinguish infants with and without FI based on their gastric myoelectrical responses to feeding.

### Analytical Development of the Study

In adults, EGG has been largely abandoned in clinical practice due to its limited reproducibility and diagnostic accuracy. Surface recordings often suffer from poor signal penetration, interference from adipose tissue, and uncertainty in electrode placement caused by greater abdominal thickness. In contrast, in infants, the thin abdominal wall and minimal subcutaneous fat allow better transmission of gastric slow-wave signals and more consistent electrode positioning directly over the stomach. These factors markedly improve the signal-to-noise ratio and electrode placement accuracy. Consequently, the application of EGG in premature infants represents a novel and promising approach, where the limitations stated above are greatly minimized and meaningful gastric motility information can be extracted non-invasively.

Traditional EGG parameters, such as absolute power, dominant frequency, or percentage of normogastria, often vary widely between individuals and recording sessions, making inter-subject comparisons unreliable. The present study introduced a simple and easy-to-quantify strategy, namely, using power spectral density ratio (PSDR) and mean power ratio (mPR) metrics, which normalize each infant's gastric response to its own baseline, thereby minimizing variability due to electrode impedance, skin thickness, or absolute amplitude scaling. These ratio-based measures emphasize relative modulation of feeding-stimulated gastric activity, providing superior sensitivity to physiological changes with interpretable underlying mechanisms. Such normalization represents a methodological advancement that enhances the precision and comparability of neonatal EGG analysis.

Traditional comparisons have been between pre-feeding and post-feeding EGG measurements. Measurements obtained during the enteral feeding itself are not usually reported. The novelty of this study is the comparison between pre-feeding and during feeding, which may be a more useful comparison in the assessment of feeding response.

Another methodological innovation in this study is the use of a non-parametric bootstrap resampling technique to compare groups with unequal sample sizes. Bootstrapping allows the estimation of empirical confidence intervals under data imbalance. This approach ensures that statistical conclusions are data-driven and distribution-free, strengthening the validity of the observed group differences and demonstrating a rigorous analytical framework applicable to future neonatal EGG.

### Clinical Implications of the Study

Results from this study suggest that early signs of dysmotility in preterm infants may be associated with increased risk of developing feeding intolerance. If validated, electrogastrography may be a future tool to screen infants at risk for feeding intolerance, particularly pathologic feeding intolerance such as necrotizing enterocolitis, which can lead to death or significant morbidity. Such an objective screening tool will be more helpful than the current nonspecific clinical signs and symptoms of FI that lead to prolonged cessation of feeding or limitation of feeding advancement. Current feeding interventions in response to these signs and symptoms may be unnecessary and may contribute to malnutrition and poor neurodevelopmental outcomes in children who do not have FI [3]. If an infant were to screen positive for FI during the first 2 weeks of life, we may be able to target therapies and interventions to this high-risk group instead of the current practice of exposing almost all infants to unnecessary interventions, such as X-ray imaging and broad spectrum antibiotics.

### Example 6: Fecal Vitamin E is Lower in Infants with Pathologic Feeding Intolerance

In some aspects, the current disclosure also encompasses correlating fecal biomarkers from preterm babies with EGG data and/or developing standalone diagnoses method to further distinguish between PFI and DFI. Developmental feeding intolerance (DFI) results from gastrointestinal (GI) immaturity and dysmotility, whereas pathologic feeding intolerance (PFI) is associated with ileus due to sepsis, necrotizing enterocolitis (NEC), spontaneous intestinal perforation (SIP), and bowel obstruction. Non-specific clinical signs of DFI are often misinterpreted as PFI, leading to feeding cessation or limitation. Therefore, the present study was undertaken to compare differences in gut microbiota-derived metabolites between infants with DFI, PFI, and no feeding intolerance (NFI).

### Methods

Provided herein is a longitudinal cohort study of infants <34 and ≥37 weeks' gestation who underwent weekly stool collection. Infants were classified into 3 groups based on objective criteria. NFI was defined as having both criteria of sustained feeding tolerance: 1) achieved full feeds (≥120 mL/Kg/day) sustained for >5 days without parenteral nutrition and 2) growth velocity ≥10 g/Kg/day from the 1st day of full feeds until 34w6d postmenstrual age. DFI was defined as having ≤1 of the aforementioned criteria and no GI pathology while PFI was defined as having GI pathology (septic ileus, NEC Stage ≥Ila, SIP, or bowel obstruction). High resolution untargeted metabolomics was performed using mass spectrometry on stool samples. Median and variance differential analysis were performed for each group comparison (NFI vs. DFI, NFI vs. PFI, and DFI vs. PFI) and displayed as a volcano plot. Independent t-tests were conducted for each annotated ion and FDR-corrected using the Benjamini-Hochberg procedure. Principal component analysis and pathway analysis are being conducted.

### Results

94 infants (82 NFI, 6 DFI, 6 PFI) were included (Table 4).

TABLE 4
Demographics

| | NFI (n = 82) | DFI (n = 6) | PFI (n = 6) |
|---|---|---|---|
| Gestational Age, weeks | 29 (23-39) | 30 (26-33) | 26 (24-28) |
| Birthweight, Kg | 1.33 (0.45-4.25) | 1.31 (0.81-2) | 0.86 (0.44-1.15) |
| Sex | | | |
| Female | 47 (57) | 4 (67) | 3 (50) |
| Male | 35 (43) | 2 (33) | 3 (50) |
| Race/Ethnicity | | | |
| Black Non-Hispanic/Latino | 20 (24) | 4 (67) | 4 (67) |
| White Non-Hispanic/Latino | 2 (2) | 0 (0) | 1 (17) |
| Hispanic/Latino | 57 (70) | 2 (33) | 1 (17) |
| Asian | 1 (1) | 0 (0) | 0 (0) |
| Unknown/Not Reported | 2 (2) | 0 (0) | 0 (0) |
| Age of First Feed, days | 2 (1-6) | 2 (1-3) | 2 (2-3) |
| Days of Hospitalization | 72 (3-197) | 63 (15-151) | 95 (39-134) |
| Days on Parenteral Nutrition | 13 (0-125) | 11 (0-17) | 63 (32-103) |
| Central Line Days | 14 (0-124) | 10 (0-16) | 54 (8-105) |
| Days to Full Feeds | 10 (2-35) | 9 (6-14) | 17 (6-34) |
| Days to Sustained Feeding | 12 (4-34) | 10 (6-15) | 32 (7-61) |
| Tolerance (SFT) | | | |
| Number of Times NPO | 1.8 (0-10) | 1.5 (0-3) | 4.7 (1-14) |
| Number of Days NPO | 2.4 (0-18) | 1.2 (0-3) | 26 (9-66) |
| Growth Velocity, g/Kg/day | 15.2 (10-28.9) | 8.6 (6.2-9.9) | 1.7 (−10.6-13.6) |
| NEC Stage ≥ IIa | | | |
| Medical | 0 (0) | 0 (0) | 1 (17) |
| Surgical or Death | 0 (0) | 0 (0) | 3 (50) |
| Bowel Obstruction (Volvulus) | 0 (0) | 0 (0) | 1 (17) |
| Septic ileus | 0 (0) | 0 (0) | 1 (17) |
| Death | 2 (2) | 0 (0) | 2 (33) |

Mean (Range) is presented for continuous variables and n (%) for categorical variables.
Growth velocity from 1^{st} day of sustained feeding tolerance to 34 w 6 d postmenstrual age
DFI = Developmental Feeding Intolerance, PFI = Pathologic Feeding Intolerance, NFI = No Feeding Intolerance, SFT = Sustained Feeding Tolerance (ability to achieve ≥120 mL/kg/day of full enteral feeds for ≥5 days without parenteral nutrition)

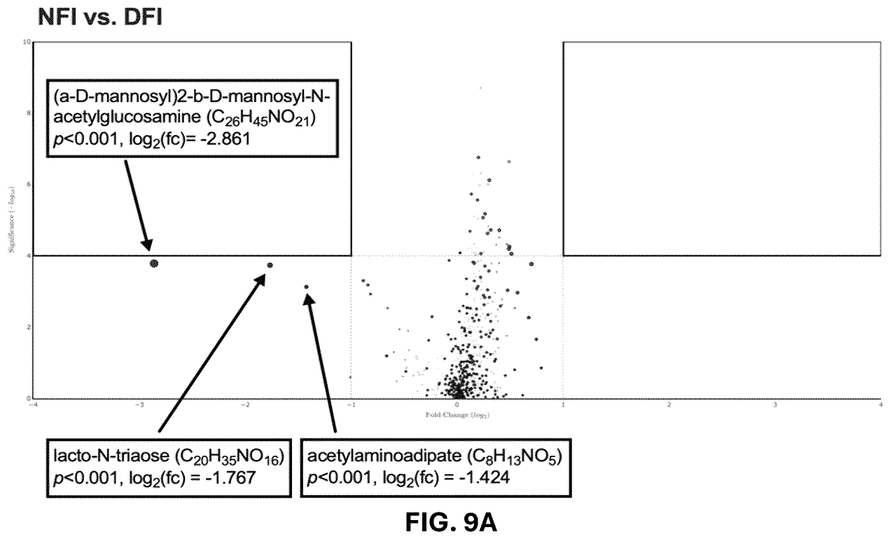

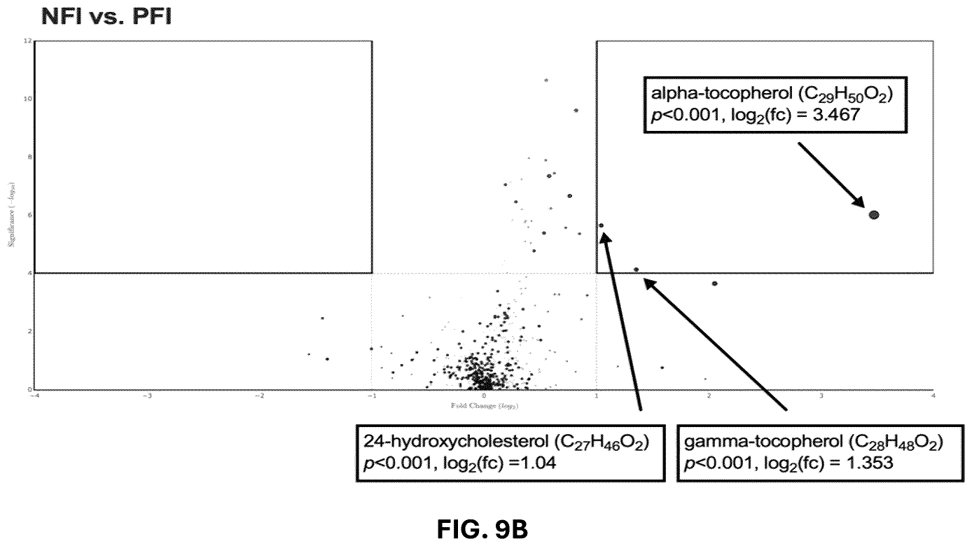

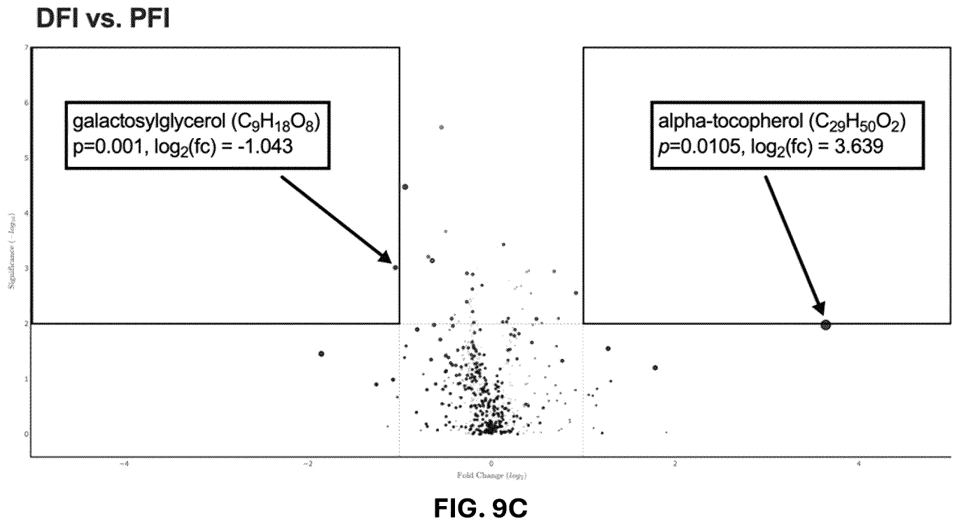

580 stool samples were analyzed. Metabolites were compared between groups with the following significant findings: The NFI group demonstrated higher alpha-tocopherol (p<0.001) and gamma-tocopherol (p<0.001) compared to PFI. The DFI group also demonstrated higher alpha-tocopherol (p=0.011) compared to PFI. See FIGS. 9A-9C for all group comparisons. Table 5 lists metabolites that were differentially abundant between groups.

TABLE 5
Microbiota-Derived Metabolites (Untargeted
Metabolomic Profiling)

| | NFI vs. DFI | NFI vs. PFI | DFI vs. PFI |
|---|---|---|---|
| alpha-tocopherol | | ↑ (p < 0.001) | ↑ (p = 0.011) |
| gamma-tocopherol | | ↑ (p < 0.001) | |
| (a-D-mannosyl)2-b- | ↓ (p < 0.001) | | |
| D-mannosyl-N- | | | |
| acetylglucosamine | | | |
| acetylaminoadipate | ↓ (p < 0.001) | | |
| lacto-N-triaose | ↓ (p < 0.001) | | |
| 24-hydroxycholesterol | | ↑ (p < 0.001) | |
| galactosylglycerol | | | ↓ (p = 0.001) |

1. A method of predicting and/or diagnosing feeding intolerance (FI) in a subject in need thereof, the method comprising:
(a) providing pre- and during feeding electrogastrography (EGG) signal data from at least one feed for the subject;
(b) obtaining mean power ratios mPRdur/pre (mean power ratio of during and pre-feeding signal) from power spectral density (PSD) obtained from during and pre-feeding EGG signals; and
(c) predicting/diagnosing feeding intolerance in the subject if the mPRdur/pre ratio is less than or equal to one.

2. The method of claim 1, wherein step (a) further comprises providing post feeding EGG data and obtaining a mPRpost/pre (mean power ratio of post/pre-feeding signal data).

3. (canceled)

4. The method of claim 1, wherein the subject is a preterm infant.

5. The method of claim 1, wherein the EGG signal data is obtained within first 1-10 weeks after birth of the subject.

6. The method of claim 5, wherein the EGG signal data is obtained within first 2 weeks after birth.

7. The method of claim 1, wherein step (b) comprises obtaining power spectral density (PSD) value within one or more of specific frequency bands comprising Bradygastria (0.5-2 cpm), Normogastria (2-4 cpm), or Tachygastria (4-9 cpm).

8. The method of claim 7, wherein step (b) comprises obtaining power spectral density (PSD) over all of the specific frequency bands (0.5-9 cpm).

9. The method of claim 1, further comprising preprocessing of the EGG data before performing step (b).

10. The method of claim 9, wherein the preprocessing of EGG data comprises one or more of:
(a) down sampling the data to 500 Hz to obtain a down sampled time series;
(b) applying a third-order polynomial fit to detrend the data and capture temporal trends in the down sampled time series;
(c) subtracting the fitted trend from the down sampled time series to obtain a detrended time series; or
(d) filtering the detrended time series using a low-pass filter at any one or more of cut off frequency in the range of 0.3 Hz to 1 Hz to mitigate potential filtering-induced phase shifts.

11. The method of claim 10, wherein the detrended signal is low-pass filtered at 0.37 Hz (~22.2 cpm) using a zero-phase second-order Butterworth filter.

12. The method of claim 1, wherein the PSD has a frequency resolution of less than 4 mHz.

13. The method of claim 1, further comprising classifying FI into pathological FI (PFI) or developmental FI (DFI).

14. A method of treating a preterm infant in need thereof for a feeding intolerance (FI) comprising:
I. determining a mean power ratio mPRdur/pre using the steps of:
(a) providing pre- and during feeding electrogastrography (EGG) signal data from at least one feed for the preterm infant; and
(b) obtaining mean power ratios mPRdur/pre from power spectral density (PSD) obtained from during and pre-feeding EGG signals, and
II. administering a treatment to the preterm infant, wherein the preterm infant has been determined to have the mPRdur/pre ratio of less than or equal to one.

15. The method of claim 14, wherein step (a) further comprises providing post feeding EGG data and obtaining a mPRpost/pre (mean power ratio of post/pre-feeding signal data).

16. The method of claim 14, wherein the treatment comprises any one or more of minimal enteral nutrition (MEN), slow advancement of feeds, use of human milk, fortified feeds, parenteral nutrition, hydrolyzed or elemental formulas, prokinetic agents, probiotics, prebiotics, nutritional supplements, upright or lateral positioning, probiotics, avoidance of unnecessary antibiotics, gastric residual monitoring, continuous vs. bolus feeding adjustments, abdominal massage, non-nutritive sucking, thickened feeds for reflux, acid suppressants in severe gastroesophageal reflux disease (GERD), bowel rest (NPO), intravenous fluids, broad-spectrum antibiotics, surgical intervention for necrosis or obstruction, or any combination thereof.

17.-22. (canceled)

23. A computer implemented method for predicting/diagnosing feeding intolerance (FI) in a subject in need thereof, the method comprising:
(a) providing pre- and during feeding electrogastrography (EGG) signal data from at least one feed for the subject;
(b) obtaining mean power ratios mPRdur/pre from power spectral density (PSD) obtained from during and pre-feeding EGG signals; and
(c) predicting/diagnosing feeding intolerance in the subject if the mPRdur/pre ratio is less than or equal to one;
(d) predicting/diagnosis no feeding intolerance (NFI) in the subject if the mPRdur/pre ratio is greater than one.

24. The method of claim 23, wherein step (a) further comprises providing post feeding EGG data and obtaining a mPRpost/pre (mean power ratio of post/pre-feeding signal data).

25. A system for predicting/diagnosing feeding intolerance (FI) in a subject, the system comprising:
(a) an EGG machine;
(b) a memory; and
(c) a processor operable to execute instructions stored in the memory to determine mean power ratios mPRdur/pre from power spectral density (PSD) obtained from during and pre-feeding EGG signals from the subject.

26. The method of claim 1, further comprising comparing the mPRdur/pre ratio to reference values using bootstrap resampling with replacement to generate empirical confidence intervals.

27. The method of claim 26, wherein the bootstrap resampling comprises at least 1,000 iterations, and wherein empirical confidence intervals are defined by the 2.5th and 97.5th percentiles of the bootstrap distribution.
